(19) United States
(12) Patent Application Publication
(10) Pub. No.: US 2019/0338362 A1
(43) Pub. Date: Nov. 7, 2019

(54) METHODS FOR NON-INVASIVE PRENATAL DETERMINATION OF ANEUPLOIDY USING TARGETED NEXT GENERATION SEQUENCING OF BIALLELIC SNPS

(71) Applicant: MedTimes Molecular Laboratory Limited, Kwai Chung (HK)

(72) Inventors: Chor Wing Jacqueline TAM, Kwai Chung (HK); Yee Man CHAN, Kwai Chung (HK); Chun Kin CHOW, Kwai Chung (HK)

(21) Appl. No.: 16/446,818

(22) Filed: Jun. 20, 2019

Related U.S. Application Data
(60) Provisional application No. 62/772,639, filed on Nov. 29, 2018.

(51) Int. Cl. C12Q 1/6883; G16B 30/00; G16B 20/20

(52) U.S. Cl. CPC C12Q 1/6883; C12Q 2600/156; G16B 20/20; G16B 30/00

(57) ABSTRACT

This invention provides methods for non-invasive prenatal testing (NIPT) for determining the probability of aneuploidy in a fetus. The present invention comprises quantification and analysis of autosomal single nucleotide polymorphisms (SNPs) using platforms capable of absolute or relative quantification to determine the probability of aneuploidy in the fetus. In one embodiment, the present methods comprise obtaining a blood sample containing cell-free DNA from a pregnant woman, using the extracted DNA to prepare a library of nucleic acids encompassing a plurality of biallelic autosomal single nucleotide polymorphisms (SNPs) of interest (i.e., target SNPs) using a target enrichment approach, performing targeted next-generation sequencing (NGS) using the library prepared, obtaining the allele counts of the target SNPs in the cell-free DNA and determining the probability of aneuploidy in a fetus.

### CROSS-REFERENCE TO RELATED APPLICATIONS

This patent application claims the benefit of U.S. Provisional Patent Application No. 62/772,639, filed Nov. 29, 2018, which is incorporated herein by reference.

### FIELD OF THE INVENTION

This invention is related to the field of non-invasive prenatal testing (NIPT) for determining the probability of aneuploidies. Specifically, this invention is related to non-invasive prenatal determination of trisomy 13 (T13), trisomy 18 (T18) and trisomy 21 (T21).

### BACKGROUND OF THE INVENTION

Pregnant women are generally advised to conduct tests for the detection of fetal chromosomal abnormality in 6-12 gestational weeks. The goal for the test is to identify the possibility that the fetus will develop aneuploidy (an abnormal number of chromosomes). It has been confirmed that Trisomy 21 (T21) will result in Down Syndrome, Trisomy 18 (T18) may lead to Edwards Syndrome, Trisomy 13 (T13) may give rise to Patau Syndrome. Early prenatal test on the fetus can open an option for parents to consider abortion when fetuses are found to be at risk for a chromosomal abnormality. It is also a way to check whether or not the baby is at risk for a chromosomal abnormality.

Unlike paternity tests which can be conducted after birth, trisomy tests are usually conducted prenatally. Traditional methods for identifying aneuploidy are invasive, among which Chorionic Villus Sampling (CVS) and Amniocentesis are the most common strategies. CVS involves inserting a needle through the mother's cervix or abdomen into the uterus for placental tissue (i.e. chorionic villus) sampling to obtain fetal DNA. It can be performed during the 10th to 14th gestational weeks and its accuracy can reach 99%. On the other hand, amniocentesis involves sampling fetal tissues from the amniotic sac surrounding the fetus. However, both procedures involve intruding the womb and carry a 1-2% miscarriage risk because of infection and amniotic fluid leakage.

An increasingly popular approach for aneuploidy detection is non-invasive prenatal testing (NIPT). Without invading the womb, NIPT merely requires the analysis of circulating cell-free fetal DNA (cffDNA) from a maternal blood sample. The discovery of cell-free fetal DNA in maternal blood in 1997 by Dr. Dennis Lo paved the way to the development of NIPT. Over the past decade, non-invasive prenatal testing or diagnosis methods have been developed to analyze fetal genetic material, and further employed in various clinical applications such as parentage relationship testing, fetal sex determination and fetal aneuploidy screening. NIPT is much less invasive than CVS and amniotic tissue-based methods because it requires only the peripheral blood of the mother, and can be done as early as on the 6th week of gestation.

cffDNA is released from biological materials of embryonic origin into the blood stream of the mother as small fragments between 150-200 base pairs. It is generally believed that cffDNA is derived from multiple tissue sources during pregnancy including placenta or fetal membranes, fetal hematopoietic cells, apoptosis or necrosis of cells and organs. Detection of cffDNA is ideally conducted during pregnancy since the cffDNA will rapidly vanish after child birth. The quantities and quality of cffDNA in maternal circulation is determined by factors such as maternal body mass index, gestational age, fetal clinical status and type of gestation (singleton or multiple).

One of the main challenges of NIPT lies in the separation of fetal DNA from maternal DNA for accurate fetal genotype identification, given that cffDNA only represents a tiny amount of DNA in the maternal blood. Determination of fetal genotype based on cffDNA analysis requires amplification such as using polymerase chain reaction (PCR) and comparison of maternal and fetal genotype. It has been demonstrated that cffDNA accounts for only 2-20% of total cell-free DNA in the maternal circulation so capturing cffDNA sequence from a maternal sample requires highly delicate and sensitive means and methods capable of absolute quantification.

The field currently uses two approaches for genotype determination. The first one is Whole Genome Sequencing (WGS), which decodes every single base pair of the genome sequence. Because of the massive size of the genome, WGS usually takes a longer time before the required genotype data can be acquired and processed. Examples for WGS include Sanger Sequencing and Massively Parallel Shotgun Sequencing (MPSS). The second and increasingly preferred approach is the target enrichment approach. Unlike WGS, the target enrichment approach only captures regions of interest for sequencing and therefore its sample size is much reduced and permits a much quicker turnaround time as compared to WGS. As explained herein, the target enrichment approach involves the use of specifically designed primers or probes in order to recognize genomic regions of interest followed by amplification of these regions in order to achieve more accurate and consistent sequencing results.

In target enrichment approach, library preparation prior to sequencing is essential. At the time of the present invention, library preparation strategies fall into two main categories, which are hybridization-based library preparation and amplicon-based library preparation. Generally speaking, both categories comprise standard procedures of adapter and index ligation, target enrichment and post-enrichment PCR amplification. This leads to another challenge for NIPT, which is the preservation of DNA quality during different stages of library preparation to ensure an accurate and sensitive result.

Several types of genotypic data are typically used in the field. Short tandem repeat-based (STR-based) methods require less genomic sequencing since they target specific loci, but their sensitivity is relatively low because STR signals from the fetus are often masked by those from the mother. Over the years, improvements of the STR technique have been made. For example, studying STR on the Y-chromosome increases the accuracy and sensitivity but such methods are only applicable to male fetuses and inevitably require sex determination prior to NIPT. The present invention takes another approach. This approach is based on examining Single Nucleotide Polymorphisms (SNPs) and it allows selective examination of only the base pairs of interest selected from approximately 3 billion base pairs within the normal human genome. Instead of sequencing the whole genome before selecting genotypes of interest, the SNP approach selects the base pairs of interest with specifically designed primers and probes at the library preparation stage which precedes sequencing. Factors such as linkage equilibrium/disequilibrium and distribution of genotypes of particular SNPs in a population need to be taken into account when choosing the SNP targets.

### SUMMARY OF THE INVENTION

This invention provides methods and materials for non-invasive prenatal detection of aneuploidy. This invention also provides methods and materials for non-invasive prenatal testing (NIPT) for determining the probability of aneuploidy in a fetus.

In one embodiment, this invention provides methods and materials for non-invasive prenatal detection of trisomy 13 (T13), trisomy 18 (T18) or trisomy 21 (T21). In one embodiment, this invention provides methods and materials for non-invasive prenatal testing (NIPT) for determining the probability of trisomy 13 (T13), trisomy 18 (T18) or trisomy 21 (T21) in a fetus.

In one embodiment, the present invention comprises quantification and analysis of autosomal single nucleotide polymorphisms (SNPs) using platforms capable of absolute or relative quantification to identify chromosomal imbalance or nucleic acid sequences that cause chromosomal disorders.

In one embodiment, this invention provides a method for non-invasive prenatal testing (NIPT) for determining the probability of aneuploidy in a fetus, wherein the method comprises obtaining a blood sample containing cell-free DNA from a pregnant woman, using the extracted DNA to prepare a library of nucleic acids encompassing a plurality of biallelic autosomal single nucleotide polymorphisms (SNPs) of interest (i.e., target SNPs) using a target enrichment approach, performing targeted next-generation sequencing (NGS) using the library prepared, obtaining the allele counts of the target SNPs in the cell-free DNA and determining the probability of aneuploidy in a fetus.

In one embodiment, the methods of the current invention comprise collection of a blood sample from a pregnant woman, extraction of cell-free DNA from the maternal sample; optionally, quantification of the extracted cell-free DNA, preparation of a library encompassing target SNPs using a target enrichment approach (i.e., hybridization-based or amplicon-based approach); performing next-generation sequencing (NGS) of the prepared library, determining genotypes and allele counts of target SNPs using a platform capable of quantitation of target nucleic acid sequences; obtaining aneuploidy statistics comprising an aggregate fetal fraction and chromosomal abnormality probability based on the genotypes and allele counts of fetal autosomal SNPs associated with the aneuploidy, and determining the probability of aneuploidy in the fetus based on the aggregate fetal fraction and the aneuploidy probability.

In one embodiment, the preparation of hybridization-based library comprises end repairing and A-tailing of the extracted cell-free DNA, adapter and index ligation, pre-enrichment PCR amplification, target enrichment by hybridizing the amplified DNA to probes designed to capture SNPs of interest, and post-enrichment PCR amplification. In some embodiments, DNA fragmentation may be performed prior to end repairing and A-tailing of the extracted cell-free DNA.

In one embodiment, the preparation of amplicon-based library comprises end repairing and A-tailing of the extracted cell-free DNA, adapter and index ligation, enrichment of SNPs of interest by selective amplification using target-specific primers, i.e. primers designed to amplify regions encompassing target SNPs and post-enrichment PCR amplification. In some embodiments, DNA fragmentation may be performed prior to end repairing and A-tailing of the extracted cell-free DNA.

In one embodiment, the aneuploidy is trisomy 13 (T13), trisomy 18 (T18) or trisomy 21 (T21).

In one embodiment, the present method is capable of determining whether the aneuploidy is a maternal or a paternal derived trisomy.

### BRIEF DESCRIPTION OF THE DRAWING

FIG. 1 is a flowchart illustrating a non-invasive prenatal testing (NIPT) for determining the probability of fetal aneuploidy according to one embodiment of the present invention.

FIG. 2 is a flowchart illustrating a system for determining the probability of fetal trisomy according to one embodiment of the present invention.

FIG. 3 is another flowchart illustrating a system for determining the probability of fetal trisomy according to one embodiment of the present invention.

FIG. 4 is a diagram showing the preparation of a library using a hybridization-based approach according to one embodiment of the present invention.

FIG. 5 is a diagram showing the preparation of a library using an amplicon-based approach according to one embodiment of the present invention.

### DETAILED DESCRIPTION OF THE INVENTION

This invention provides methods and materials for non-invasive prenatal detection of aneuploidy. This invention also provides methods and materials for non-invasive prenatal testing (NIPT) for determining the probability of aneuploidy in a fetus.

In one embodiment, this invention provides methods and materials for non-invasive prenatal detection of trisomy 13 (T13), trisomy 18 (T18) or trisomy 21 (T21). In one embodiment, this invention provides methods and materials for non-invasive prenatal testing (NIPT) for determining the probability of trisomy 13 (T13), trisomy 18 (T18) or trisomy 21 (T21) in a fetus.

In one embodiment, the present invention comprises quantification and analysis of autosomal single nucleotide polymorphisms (SNPs) using platforms capable of absolute or relative quantification to identify chromosomal imbalance or nucleic acid sequences that cause chromosomal disorders.

In one embodiment, this invention provides a method for non-invasive prenatal testing (NIPT) for determining the probability of aneuploidy in a fetus, wherein the method comprises obtaining a blood sample containing cell-free DNA from a pregnant woman, using the extracted DNA to prepare a library of nucleic acids encompassing a plurality of biallelic autosomal single nucleotide polymorphisms (SNPs) of interest (i.e., target SNPs) using a target enrichment approach, performing targeted next-generation sequencing (NGS) using the library prepared, obtaining the allele counts of the target SNPs in the cell-free DNA and determining the probability of aneuploidy in a fetus.

In one embodiment, the methods of the current invention comprise collection of a blood sample from a pregnant woman, extraction of cell-free DNA from the maternal sample; optionally, quantification of the extracted cell-free DNA, preparation of a library encompassing target SNPs using a target enrichment approach (i.e., hybridization-based or amplicon-based approach); performing next-generation sequencing (NGS) of the prepared library, determining genotypes and allele counts of target SNPs using a platform capable of quantitation of target nucleic acid sequences; obtaining aneuploidy statistics comprising an aggregate fetal fraction and chromosomal abnormality probability based on the genotypes and allele counts of fetal autosomal SNPs associated with the aneuploidy, and determining the probability of aneuploidy in the fetus based on the aggregate fetal fraction and the aneuploidy probability.

In one embodiment, the preparation of hybridization-based library comprises end repairing and A-tailing of the extracted cell-free DNA, adapter and index ligation, pre-enrichment PCR amplification, target enrichment by hybridizing the amplified DNA to probes designed to capture SNPs of interest, and post-enrichment PCR amplification. In some embodiments, DNA fragmentation may be performed prior to end repairing and A-tailing of the extracted cell-free DNA.

In one embodiment, the preparation of amplicon-based library comprises end repairing and A-tailing of the extracted cell-free DNA, adapter and index ligation, enrichment of SNPs of interest by selective amplification using target-specific primers, i.e. primers designed to amplify regions encompassing target SNPs, and post-enrichment PCR amplification. In some embodiments, DNA fragmentation may be performed prior to end repairing and A-tailing of the extracted cell-free DNA.

In one embodiment, the aneuploidy is trisomy 13 (T13), trisomy 18 (T18) or trisomy 21 (T21).

In one embodiment, the present method is capable of determining whether the aneuploidy is a maternal or a paternal derived trisomy.

In one embodiment, methods described herein are non-invasive because the methods do not involve invading the womb to obtain fetal genetic materials.

In one embodiment, the present invention provides a non-invasive prenatal testing (NIPT) for determining the risk or probability of aneuploidy in a fetus. In one embodiment, fetal aneuploidy is detected by sequencing a plurality of selected autosomal biallelic SNPs on the chromosome in question. In one embodiment, the chromosome is chromosome 13, 18 or 21.

In one embodiment, the significance of the present invention is the utilization of hybridization-based and amplicon-based library preparation for the enrichment of genetic material, and the utilization of NGS platforms for sequencing a targeted selection of SNPs, thereby enabling aneuploidy detection associated with abnormally high chromosomal concentrations. The specific kind of aneuploidy (e.g. T13, T18 or T21, and whether it is maternally or paternally derived) can be determined based on the reads of specific chromosomes through a set of bioinformatic calculations.

In one embodiment, the present invention provides an innovative and cost-effective method for aneuploidy determination which is more efficient, sensitive and precise than any of the traditional aneuploidy tests existing at the time of the invention. As detailed herein, the present invention comprises and optimizes various steps from sampling to genotypic analysis of the fetus chromosome in question. The invention is distinctive from known approaches because it directly determines and analyzes circulating cell-free fetal DNA (cffDNA) from maternal whole blood samples. The approach obtains a value of counts for a selected set of SNPs from the maternal plasma cell-free DNA using platforms that are capable of targeted sequencing with DNA markers such as indexes and Unique Molecular Identifiers (UMIs), and quantitation of target nucleic acid sequences. In one embodiment, where NGS is used, various mathematical calculations are used to compare the SNP allele counts from the collected data with those in chromosomal disorder models so as to ascertain the likelihood that the fetus has the chromosomal disorder. In one embodiment, the counts obtained for the selected set of SNPs are absolute and sequencing approaches that are capable of absolute quantitation are used.

In one embodiment, the present invention provides a proprietary system or algorithm for analyzing the data obtained from the sequencing and thereby determining the probability of having a particular aneuploidy in the fetus.

In one embodiment, the present algorithm is an extension to the algorithm of Goya et al. [1] which examines sequencing data to determine single nucleotide variations in tumor DNA. For example, when fetal fraction (ff)=10%, euploid fetus should give possible allele frequencies of 0, 0.05, 0.45, 0.5, 0.55, 0.95 and 1.0 (Table 3). In contrast, maternal trisomy with 100% isodisomy (i.e., both copies of a chromosomal set being inherited from one parent only) should have possible allele frequencies of 0, 0.047619, 0.4285714, 0.47619, 0.52381, 0.571428, 0.95238 and 1.0. These two models give different likelihoods, and the ratio between the two likelihoods can be converted into a probability of a particular fetal aneuploidy if the maternal age and gestational week are considered. Overall, the present algorithm takes into account a set of factors including maternal age and gestational week and allele frequencies of the two alleles of biallelic SNPs for different aneuploidy models, and translate likelihood ratios of these aneuploidy models into posterior probability of a particular aneuploidy.

At the time of this invention, there are methods which require determination of genotypes of the fetus, the biological mother and the biological father for aneuploidy determination. In contrast, the present invention does not require any genetic information about the biological father yet is able to produce accurate and sensitive results based on the maternal and fetal genotypes alone by using the proprietary methodology and algorithm described herein. It is found that the present method can detect fetal aneuploidy as early as in the seventh gestational week and is able to determine whether the fetal aneuploidy is a maternally or a paternally derived trisomy. Therefore, some embodiments of the present invention can provide an aneuploidy test with high accuracy, sensitivity and efficiency at a significantly lower cost as compared to existing methods.

### Definition of Terms

As used herein, single nucleotide polymorphism (SNP) refers to the variation of a single nucleotide at a specific location in a nucleic acid sequence, e.g. when some individuals have one nucleotide at a specific location within their genome, while others have a different nucleotide at the corresponding location. If more than 1% of a population does not carry the same nucleotide at a specific position in the DNA sequence, then this variation can be classified as a SNP. SNPs could occur at either coding or non-coding regions of the DNA sequence.

As used herein, autosomal single nucleotide polymorphism refers to a SNP that is not located on any sex chromosome.

As used herein, sequence refers to a nucleotide sequence of any length or type or genetic information in the DNA molecule.

As used herein, locus refers to a specific location on a chromosome, which could be of any length from a few base pairs to a mega base-size region containing a large gene family.

As used herein, allele refers to a variant form of a gene that is located at the same locus on the chromosome and is responsible for hereditary variation. For diploid organisms, such as humans, an individual normally has two alleles at each locus, with one allele inherited from the mother and another one inherited from the father.

As used herein, polymorphic gene or locus refers to a gene or locus which has two or more alleles within a population.

As used herein, genotype refers to the genetic makeup of an organism. It can also refer to a particular sequence of base pairs that comprises a chromosome, an allele in a gene or locus that is carried by an organism. For example, each pair of alleles represents the genotype of a specific gene.

As used herein, aneuploidy refers to the presence of an abnormal number of chromosomes in a cell. A cell that has either greater or smaller number of chromosomes than that of the wild type is called an aneuploid cell. Usually, an aneuploid cell's chromosome set differs from wild type by only one or a small number of chromosomes. A cell with the correct number of chromosomes is called a euploid cell.

As used herein, maternal plasma refers to the non-cellular portion of the blood of a pregnant woman. It is mostly made up of water and contains dissolved proteins, glucose, electrolytes, hormones, carbon dioxide, and oxygen. The maternal plasma also contains cell-free fetal and cell-free maternal DNA.

As used herein, nucleic acid is a molecule that is made of nucleotides, including ribonucleic acid (RNA) and deoxyribonucleic acid (DNA). As used herein, nucleic acids can be from any species, of any length (e.g. oligonucleotide or polynucleotide), naturally-occurring or synthetic (i.e., artificially made and containing natural and/or non-natural nucleotides), linear, circular or in other configuration, a mixture of DNA or RNA and so on.

As used herein, amplification refers to an increase in the number of copies of a particular DNA fragment through replication of the segment by any applicable method such as polymerase chain reaction (PCR).

As used herein, polymerase chain reaction (PCR) refers to a process which amplifies specific DNA segments using polymerase.

As used herein, primer refers to a short strand of nucleic acid that serves as a starting point for synthesis of nucleic acid. For example, a primer is required in DNA replication by DNA polymerases, which add new nucleotides to an existing strand of nucleic acid.

As used herein, probe refers to a single stranded nucleic acid which is 100% or sufficiently complementary to a target sequence so that it can be used to detect a target sequence among a mixture of other single-stranded nucleic acid molecules or to differentiate a target sequence from other nucleic acid molecules.

As used herein, non-invasive prenatal testing (NIPT) refers to a test procedure that does not involve any breakage of skin of a fetus, removal of tissue from the fetus, or contact with the mucous membrane or internal body cavity of a fetus in a pregnant woman.

As used herein, fetal genotype refers to the genetic makeup of the fetus. It could also refer to the alleles carried by the fetus at a specific locus.

As used herein, linkage disequilibrium refers to the non-random association of alleles at different loci in a given population. Loci are said to be in linkage disequilibrium when the frequency of association of their different alleles is higher or lower than what would be expected if the loci were independent and associated randomly.

As used herein, target enrichment approach refers to the selection of alleles from the chromosome of interest with specific designs of primers or probes carrying sequences that are capable of recognizing the locus of interest or distinguishing the locus of interest from that which is not of interest.

As used herein, target-specific primer refers to a primer comprising nucleic acid sequence which is capable of recognizing a target locus or distinguishing the target locus from non-target locus. For instance, the primer may comprise a sequence within the target locus, or comprise a sequence that will bind to a particular region of the genome leading to amplification of the target locus.

As used herein, target-specific probe refers to a probe comprising nucleic acid sequence which is capable of recognizing a target locus or distinguishing the target locus from non-target locus. For instance, the probe may comprise a sequence within the target locus, or comprise a sequence that will bind to a particular region of the genome leading to hybridization between the probe and the target locus.

As used herein, targeted next-generation sequencing or NGS refers to next-generation technology that sequences nucleic acids obtained from a library of nucleic acids encompassing nucleic acid sequences of interest (i.e., the target sequences). The library includes libraries prepared according to the description of this invention.

As used herein, percentage of isodisomy refers to percentage of the two chromosomes in a gamete cell that come from one of the two chromosomes of one parent. For example, when the percentage of isodisomy of the two chromosomes in a gamete cell is 100%, the two chromosomes are 100% identical and come from one of the two chromosomes of one parent. This situation can occur when there was no chromosomal crossover in Meiosis I and a non-disjunction event occurred in Meiosis II. On the other end of the spectrum, when there was no crossover in Meiosis I and a non-disjunction event occurred in Meiosis I, a gamete cell would form with 0% isodisomy (in other words 100% heterodisomy). Usually chromosomal crossover does occur in Meiosis I, so a typical disomic gamete cell has a percentage of isodisomy between 0% and 100%.

As used herein, allele count refers to the frequency of a particular allele obtained from a platform or method capable of quantitation of the allele, including but is not limited to any kind of sequencing platform and digital PCR. Generally, allele count represents the quantity of a particular allele at a particular locus in the sample being tested.

As used herein, reference allele refers to the nucleotide on the positive strand of the reference genome, e.g. in human reference genome builds hg19 or hg38, for a SNP that has a certain chromosomal position.

As used herein, alternate allele refers to any allele other than the reference allele. For a biallelic SNP, alternate allele refers to the second allele of a biallelic SNP.

As used herein, base quality refers to the error rate of a nucleotide sequenced by a sequencing machine. It is a value reported by the sequencing machine.

As used herein, sequencing depths refers to the number of sequencing reads generated by a sequencer that are aligned to the reference genome. Sequencing depth at a biallelic SNP refers to the number of reads aligned to that position

As used herein, minor allele frequency refers to the smaller value of the reference allele frequency and the alternate allele frequency in a population.

As used herein, fetal fraction (ff) refers to the proportion of cell-free DNA that is fetal in origin in the maternal sample. Generally, fetal fraction can be an actual value determined by calculating the actual proportion of cell-free fetal DNA in the maternal sample, or an estimated value determined using an appropriate algorithm based on the data of the sample in question.

### Overview

In one embodiment, the present invention provides a non-invasive prenatal aneuploidy test for detection of fetal aneuploidy or determining the probability of fetal aneuploidy. In one embodiment, the fetal aneuploidy to be tested is the existence of an extra copy of chromosome 13, 18 or 21 in the cells of a fetus. In one embodiment, the present invention provides a non-invasive prenatal test for determining the probability or detecting the existence of one or more of trisomy 13 (T13), trisomy 18 (T18) and trisomy 21 (T21) by comparing the allele counts of SNPs of the aforesaid chromosomes of the fetus in question and those of models of said trisomy.

The present non-invasive prenatal aneuploidy test may include one or more of the following steps:
1) obtaining a test sample from a pregnant woman, the sample comprises cell-free fetal DNA and cell-free maternal DNA;
2) enriching a plurality of target sequences in the cell-free fetal DNA and cell-free maternal DNA, where the target sequences comprise a plurality of biallelic autosomal single nucleotide polymorphisms (SNPs) of interest;
3) amplifying the enriched target sequences;
4) sequencing some or all of said target sequences; and
5) determining the probability that the fetus suffers from an aneuploidy by analyzing allele counts for the plurality of SNPs.

In one embodiment, the present non-invasive prenatal aneuploidy test may include one or more of the following steps:
1) collecting a peripheral blood sample from a pregnant woman (the mother);
2) extracting cell-free DNA from the sample;
3) determining the concentration of the extracted cell-free fetal DNA and cell-free maternal DNA;
4) preparing a library of nucleic acids comprising a plurality of biallelic autosomal single nucleotide polymorphisms (SNPs) of interest (i.e., target SNPs) using the extracted cell-free DNA, where sequences comprising the target SNPs are selectively enriched and amplified;
5) performing next-generation sequencing (NGS) of the enriched and amplified sequences from the library;
6) filtering the plurality of target SNPs with respect to their informative value;
7) obtaining aneuploidy statistics comprising an aggregate fetal fraction and an aneuploidy probability based on the allele counts of the target SNPs obtained from the sequencing step;
8) determining whether aneuploidy likely exists in the fetus or not.

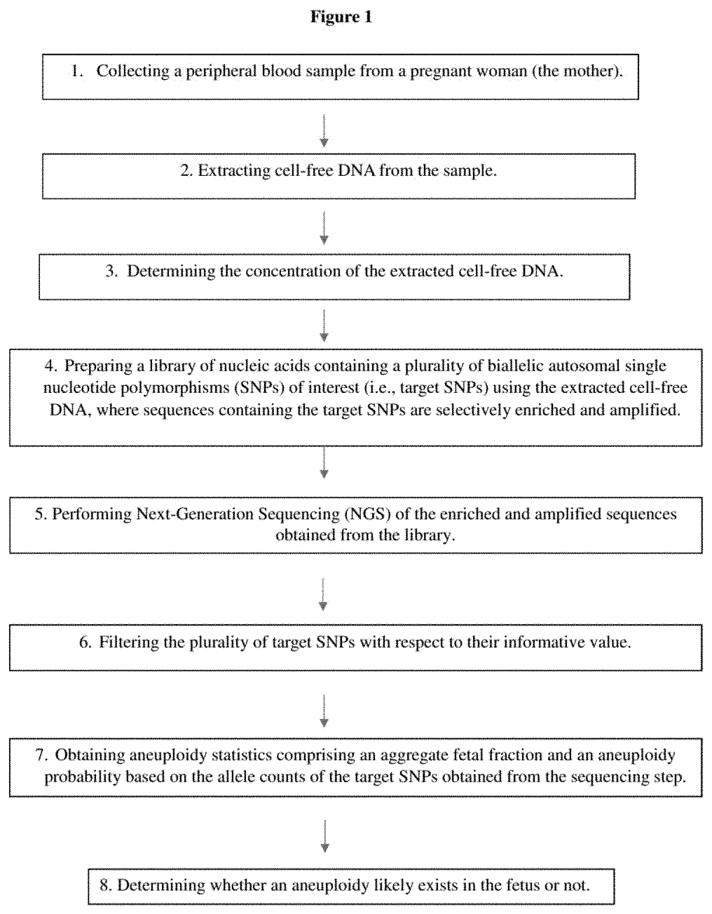

FIG. 1 is a flowchart illustrating a non-invasive prenatal testing (NIPT) for determining the probability of fetal aneuploidy according to one embodiment of the present invention.

### Collection of Samples and Extraction of Nucleic Acids

In one embodiment, the present method comprises a step of obtaining a blood sample from the pregnant mother as well as a step of extracting the genetic materials from the sample from the pregnant mother. In one embodiment, the blood sample is a peripheral blood sample.

Circulating cell-free DNA from maternal whole blood can be extracted by means of commercially available cell-free DNA extraction kits on either manual or automated extraction platforms. Examples of extraction kits include the following: GenElute™ Plasma/Serum Cell-Free Circulating DNA Purification Midi Kit, MAGMAX Cell-Free DNA Isolation Kit, QIAamp® circulating nucleic acid kit, PME free-circulating DNA Extraction Kit, MAGNA Pure Compact Nucleic Acid Isolation Kit I, Maxwell® RSC ccfDNA Plasma Kit, EpiQuik™ Circulating Cell-Free DNA Isolation Kit, NEXTprep-Mag™ cfDNA Isolation Kit, BioChain's cfPure™ Cell Free DNA Extraction Kit, NORGEN BIOTEK CORP Plasma/Serum Cell-Free Circulating DNA Purification Mini Kit, Quick-cfDNA™ Serum & Plasma Kit, MagBio Genomics cfKapture™ 21 Kit (cell-free DNA isolation kit), HIPRO CIRCULATING CELL-FREE DNA (CFDNA) ISOLATION KIT, Cell3™ Xtract cell-free DNA Extraction Kit, ALINE Cell Free DNA Isolation Kit, TruTip® Cell-free DNA Kit, InviMag® Free Circulating DNA Kit, Chemagic™ cfNA 5 k Kit, Omega Bio-tek® cfDNA extraction kit, FitAmp™ Plasma/Serum DNA Isolation Kit, MAGPURIX Cell-Free Circulating (CFC) DNA Extraction Kit, truXTRAC™ cfDNA Kits, AmoyDx® Serum/Plasma cell-free DNA Kit, IGEN cfDNA kit, XCF COMPLETE Exosome and cfDNA Isolation Kit and BIOFACTORIES' 5 min Circulating DNA Extraction Kit. One of skill in the art would understand that other commercial kits or non-commercialized methods and combinations thereof suitable for extracting cell-free DNA from maternal blood may be used.

Example 1 describes one embodiment of extraction of cell-free DNA from maternal whole blood or plasma samples using Promega Maxwell® Rapid Sample Concentrator (RSC).

### Determination of Concentration or Quantity of Nucleic Acids in a Sample

In one embodiment, the present method comprises a step of determining the concentration or quantity of the cell-free nucleic acids extracted from a maternal sample. In one embodiment, concentration or quantity of the cell-free DNA is determined by any instruments or methods that are capable of quantifying the nucleic acids including but not limited to Qubit® Fluorometer.

In one embodiment, Qubit® Fluorometer and Qubit® dsDNA High Sensitivity assay kit are used to measure the concentration or quantity of cell-free DNA extracted from the maternal sample.

Example 1 describes one embodiment of determination of the concentration of the extracted cell-free DNA using Qubit® dsDNA High Sensitivity assay kit and Qubit® instrument.

### Construction of Library Using Target Enrichment Approach

In one embodiment, the present invention comprises a step of preparing a library using a target enrichment approach. The library will then be subject to a step of next-generation sequencing (NGS).

In one embodiment, the present invention uses nucleic acid sequences which comprise sequences specific to autosomal biallelic SNPs of the target chromosome (i.e., target sequences), adapter sequences and index sequences to enrich the target sequences prior to NGS. In one embodiment, nucleic acid sequences to be used for enriching the target sequences are sequences which include the SNPs of interest or sequences which, although do not include the SNPs of interest, are able to distinguish the SNPs of interest from non-target sequences (e.g. upstream or downstream sequences of the SNPs of interest). It is appreciated that a skilled person in the art would be able to choose appropriate nucleic acid sequences that enable the implementation of this invention according to the aneuploidy in question and the description of this invention.

In one embodiment, the library is prepared using a hybridization-based approach.

In one embodiment, the library is prepared using an amplicon-based approach.

In one embodiment, the extracted cell-free DNA is treated by a fragmentation procedure to break the DNA into pieces prior to the preparation of the library. In one embodiment, the fragmentation procedure is sonication-based or enzyme-based.

### Library Preparation: Hybridization-Based

In one embodiment, a hybridization-based library preparation is used, i.e., target-specific probes are used to select and retain target sequences by probe hybridization, and the target sequences are enriched by amplification. In one embodiment, the hybridization-based approach comprises the following steps:
1. End repairing and A-tailing of the extracted cell-free DNA;
2. Ligating the cell-free DNA with adapters and index sequences;
3. Washing away non-ligated fragments;
4. Performing pre-enrichment amplification of the ligated cell-free DNA;
5. Purification of amplified sequences;
6. Selecting fragments containing target sequences by probe hybridization, wherein each of the probes used in the hybridization includes a sequence that is capable of hybridizing to a DNA fragment comprising at least one target SNP;
7. Washing away the non-selected fragments;
8. Performing post-enrichment amplification of the selected target sequences; and
9. Purification of amplified target sequences.

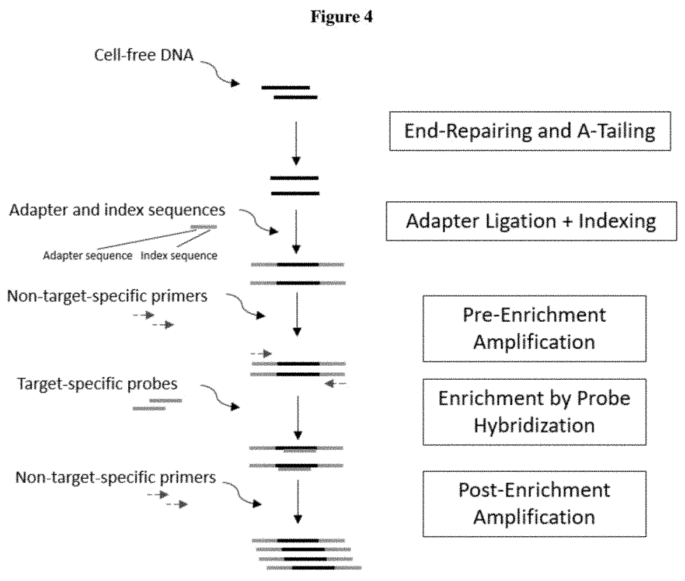

As used herein for the preparation of hybridization-based library, adapters are short stretches of synthetic DNA which enable the DNA ligated with the adapters to bind to the sequencing platform to start the process of sequencing. Index sequences are sequences specific to a sample to identify nucleic acid products derived from a particular sample, this enables a mixture of nucleic acid products from different samples to be sequenced in the same sequencer at the same time. In one embodiment, ligation of the cell-free DNA with adapters and index sequences can be done concurrently by providing a population of nucleic acids which comprises both the adapter sequence and the indexing sequence (e.g. FIG. 4). In some embodiments where sample indexing is not required, adapters comprising the adapter sequence are provided for the ligation step.

FIG. 4 depicts one embodiment of the present hybridization-based approach for library preparation.

In one embodiment, each of the probes for selecting target SNP sequences in the present hybridization-based approach comprises a sequence that is capable of hybridizing to a DNA fragment containing the SNP of interest.

It is appreciated that one skilled in the art would be able to design probes for this hybridization-based approach according to the aneuploidy in question and the description of this invention.

In one embodiment, the present hybridization approach is adapted from Roche SeqCap. In this embodiment, the hybridization-based library preparation starts with end-repairing and A-tailing of the cell-free DNA sample. This is to ensure that the delicate and fragmented cell-free DNA can easily ligate with other nucleic acid sequences such as adapters. Next, the cell-free DNA is ligated with adapters and indices. Non-ligated DNA fragments are washed away, and the DNA sample is subject to PCR amplification, after which DNA fragments are purified. This is followed by probe hybridization to capture (i.e., enrich) fragments containing the SNPs of interest. Non-hybridized DNA fragments are washed away. The DNA sample is then further amplified and purified to complete the library preparation procedure.

Example 2 describes one embodiment of the hybridization-based target capture method for use with cell-free DNA extracted from maternal plasma. Cell-free DNA was prepared using the KAPA library preparation kit and target capture was performed with custom-designed NimbleGen SeqCap EZ probes to enrich selected SNP loci located on chromosomes 13, 18 and 21. The libraries were then paired-end sequenced using an Illumina sequencer.

### Library Preparation: Amplicon-Based

In one embodiment, an amplicon-based library preparation approach is used, i.e., target-specific primers are used to selectively amplify and hence enrich the target sequences. In one embodiment, the amplicon-based approach comprises the following steps:
1. End repairing and A-tailing of the extracted cell-free DNA;
2. Ligating the cell-free DNA with platform-specific adapter sequences, index sequences and Unique Molecular Identifiers (UMIs);
3. Washing away non-ligated fragments;
4. Performing amplification of the ligated cell-free DNA using target specific primers;
5. Amplifying enriched fragments using universal primers; and
6. Purification of amplified target sequences.

In one embodiment, index sequences are added to the target sequences in the post-enrichment amplification as well as in the step of ligation.

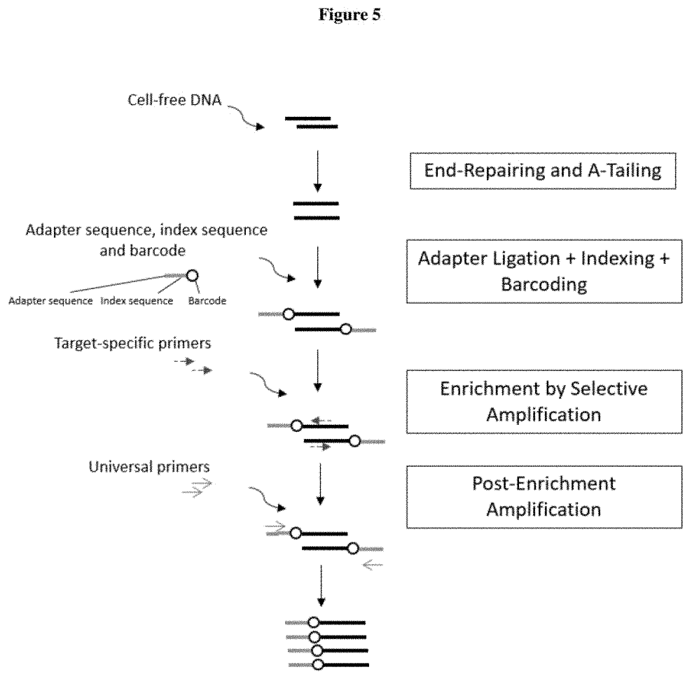

As used herein for the preparation of amplicon-based library, adapters are short stretches of synthetic DNA which enable the DNA ligated with the adapters to bind to the sequencing platform to start the process of sequencing. Index sequences are sequences specific to a sample that enable identification of nucleic acid products derived from a particular sample, and their incorporation allows a mixture of nucleic acid products from different samples to be sequenced in the same sequencer at the same time. Unique Molecular Identifiers (UMIs) are molecular tags that establish a distinct identity for each input molecule, thereby allowing to account for PCR amplification bias. In one embodiment, adapters, index sequences and barcodes such as UMIs are provided in one single nucleic acid as depicted in FIG. 5. In some embodiments, these sequences are provided separately and attached to the cell-free DNA separately (accomplished, for example, by ligation to the cell-free DNA or by amplification of the cell-free DNA).

FIG. 5 depicts one embodiment of the present amplicon-based approach for library preparation.

In one embodiment, primers for selecting target SNP sequences in the present amplicon-based approach comprise sequences specific to the target SNP sequences (i.e., sequences that are able to bind to a sample DNA fragment containing the SNP of interest hence allowing the SNP of interest to be amplified).

It is appreciated that one skilled in the art would be able to design primers for this amplicon-based approach according to the aneuploidy in question and the description of this invention.

In one embodiment, the present amplicon-based approach is adapted from QIAseq and optimized. The extracted cell-free DNA is fragmented with a fragmenting enzyme. The DNA fragments are then ligated with platform-specific adapters and indexing sequences, and barcoded with Unique Molecular Identifiers (UMIs). Non-ligated DNA fragments are washed away. The DNA sample is then subjected to targeted PCR to achieve target enrichment. The resulting indexed amplicons are further amplified in a Universal PCR using universal primers that bind to the adapters to complete the library preparation procedure.

Example 3 describes one embodiment of amplicon-based target capture method for use with cell-free DNA extracted from maternal plasma.

### Next-Generation Sequencing

In one embodiment, the present invention includes a step of performing Next-Generation Sequencing (NGS) for sequencing SNPs captured and enriched by the present hybridization-based or amplicon-based library preparations.

In one embodiment, next-generation sequencing as used herein refers to a sequencing technology capable of massively parallel sequencing of nucleic acids [2]. It generally comprises three steps: library preparation, amplification and sequencing.

In one embodiment, platform to be used for next-generation sequencing is any platform which is capable of performing next-generation sequencing.

In one embodiment, platform to be used for next-generation sequencing includes but is not limited to Illumina (MiSeq, NextSeq, HiSeq, NovaSeq), Thermo Fisher (PGM, Proton, S5), Pacific Biosciences (Sequel), and Oxford Nanopore (Minion, Gridion, Promethion).

In one embodiment, the methodology of Illumina sequencing is employed. The adapter sequence on the DNA fragments attach to the complementary oligonucleotides on the acrylamide-coated glass flow cell of the sequencing machine. Each of the bound fragments is then amplified through bridge amplification to generate clonal clusters composed of hundreds of DNA strands. These clusters form the templates for sequencing. Fluorescent-labeled nucleotides are incorporated and detected in repeated sequencing cycles to generate sequencing reads. For paired-end sequencing, both forward and reverse strands are sequenced giving forward and reverse reads as read pairs. The read pairs are aligned together for analysis. Aneuploidy is then identified based on the read information. In one embodiment, absolute quantification of the target SNPs can also be obtained from the sequencing data.

### Allele Discrimination and Quantitation of SNP Targets

In one embodiment, the present method comprises a step of collecting genotypic measurements of the cell-free DNA found in the plasma of a pregnant mother. In another embodiment, the present method comprises obtaining genotyping data for a set of autosomal SNPs of a fetus.

In one embodiment, the maternal and fetal genotypes are derived from the results of targeted sequencing of cell-free DNA described herein.

### SNP Selection Criteria for Noninvasive Prenatal Testing

In one embodiment, SNPs subject to the present invention are biallelic according to the 1,000 Genome Project.

In one embodiment, genomic position of the target SNPs is within the high confidence regions of Genome In A Bottle (GIAB) project.

In one embodiment, SNPs having minor allele frequencies greater than 0.3 for all five populations sequenced in the 1,000 Genome Project: the Africans, the Americans, the East Asians, the South Asians and the Europeans are selected.

In one embodiment, SNPs having genotype frequencies that are in Hardy Weinberg Equilibrium, i.e. p-value>=0.05 with chi-square test with one degree of freedom are selected.

In one embodiment, SNP-derived fixation index among the aforementioned five populations from the 1,000 Genome Project is <0.05.

In one embodiment, target SNPs from the same chromosome are not in linkage disequilibrium for all five populations from the 1,000 Genome Project, i.e. r²<0.1.

Filtering of SNPs with Respect to their Informative Value

In one embodiment, power in aneuploidy determination of a plurality of SNPs means the power of these SNPs to sufficiently detect the existence of an aneuploidy (e.g. a particular trisomy) in a fetus. This is determined based on the number of captured SNPs on each target chromosome and their informative values.

In one embodiment, informative value of a SNP is determined based on a number of factors, including but not limited to, sequencing depths, mapping qualities, and base quality of the two alleles at the captured SNP site. Sequencing depths reflect whether or not sufficient information is available for subsequent analysis, while mapping qualities and base quality reflect whether or not the available data is of sufficient quality to be included in the analysis.

In one embodiment, SNP which has a low informative value is removed from subsequent steps of analysis for calculating the aneuploidy probability.

### Analysis of SNP Genotyping and Quantitative Data

In one embodiment, the present invention provides a proprietary system and/or modeling method for analyzing the data obtained from sequencing and thereby determining the probability of a particular aneuploidy in the fetus. Requiring only fetal and maternal genetic information derived from the maternal sample, the present invention is robust enough to give a sensitive and accurate fetal aneuploidy test without the need for genetic information from the biological father.

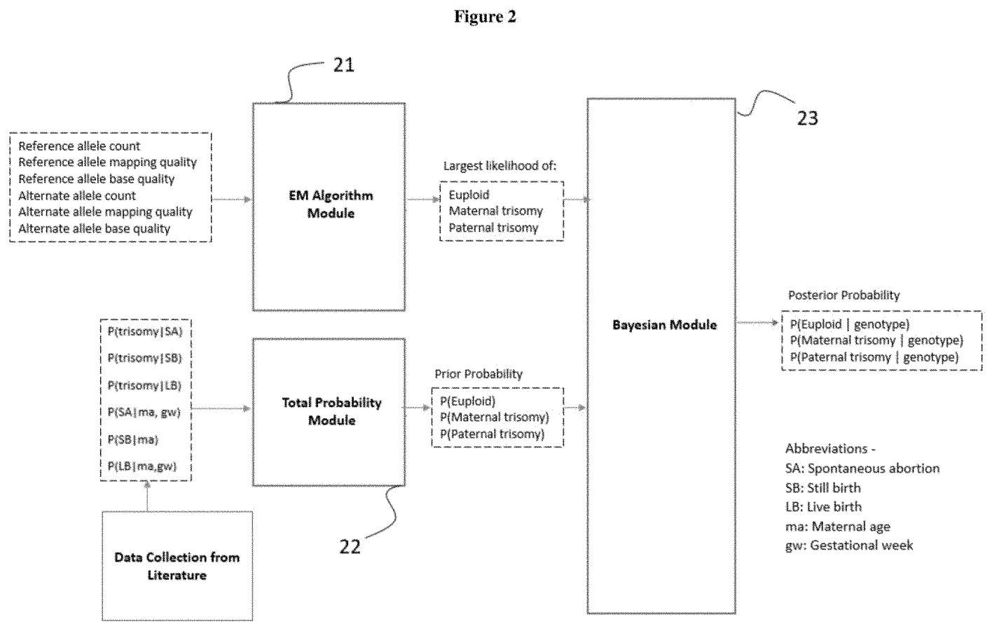

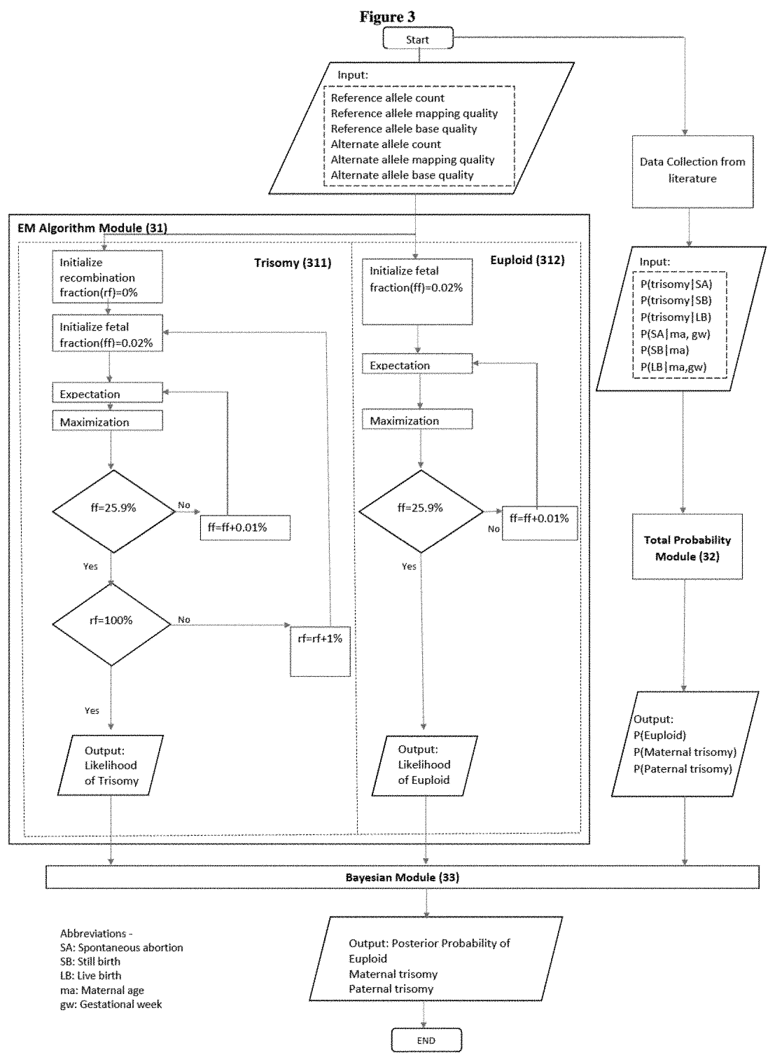

FIGS. 2-3 are two flowcharts illustrating a system for calculating the probability of fetal trisomy according to one embodiment of the present invention. It should be noted that a skilled person in the art would be able to derive a similar system for other non-trisomy aneuploidy according to the description of this invention.

In one embodiment, the present invention is used to analyze data and information about selected nucleic acid sequences, the invention comprises one or more of the following modules or steps of operating these modules: expectation-maximization (EM) algorithm module, total probability module and Bayesian module.

In one embodiment, the present invention receives a plurality of data (input) and provides a plurality of likelihood and probability parameters (output), including but not limited to those described in Table 1.

TABLE 1
Types of input and output data applicable to one embodiment of the
invention

| Input: | Output: |
|---|---|
| Allele count, mapping quality and | Largest likelihoods of (i) euploidy, |
| base quality of reference allele. | (ii) maternal trisomy, and (iii) |
| Allele count, mapping quality and | paternal trisomy |
| base quality of alternate allele. | Probabilities of (i) euploidy, (ii) |
| Conditional probabilities for | maternal trisomy, and (iii) paternal |
| aneuploidy with respect to survival | trisomy |
| probabilities, maternal age and | Posterior probabilities of (i) |
| gestational week. | euploidy, (ii) maternal trisomy, and |
| | (iii) paternal trisomy |

### Expectation-Maximization (EM) Algorithm Module

In one embodiment, the present invention provides an expectation-maximization (EM) algorithm module for estimating parameters of a Bayesian model that maximizes the likelihood of the Bayesian model. As illustrated in FIG. 2, data about SNPs of interest obtained from previous steps of sequencing are input into the EM algorithm module (21) to estimate the best possible set of mixtures of maternal-fetal genotypes at many biallelic SNPs based on the sequencing depth data, mapping quality data and base quality data obtained from sequencing. In one embodiment, data to be input into the EM algorithm module include without limitation the count, mapping quality and base quality of the reference allele, and the count, mapping quality and base quality of the alternate allele, which can be derived from the sequencing data using data processing tools available in the art, e.g. picard+GATK. In one embodiment, the EM algorithm module determines the largest likelihood of ploidy status such as euploidy, maternal trisomy and paternal trisomy.

As illustrated in FIG. 3, the expectation-maximization (EM) algorithm module (31) comprises a trisomy EM module (311) for determining the largest likelihood of a trisomic fetus and a euploid EM module (312) for determining the largest likelihood of a euploid fetus.

The trisomy EM module (311) begins with an iteration cycle using an initial recombination fraction (if) of 0% and an initial fetal fraction (ff) of 0.02%. The euploid EM module (312) begins with an iteration cycle using an initial ff of 0.02%. A brute force approach is then used to try various possible values in a series of iteration cycles each consisting of a step of expectation (i.e. missing data is estimated given the data provided and current estimate of the model parameters) and a step of maximization (i.e., the likelihood function is maximized). The value of recombination fraction (rf) refers to the completeness of recombination and the current EM algorithm module increments the if value by 1% in each cycle within a range of 0-100%, where 0% denotes no recombination and 100% denotes complete recombination. The value of fetal fraction (ff) refers to the proportion of cell-free DNA that is fetal in origin in the maternal sample, and the current EM algorithm module increments the ff value by 0.01% in each cycle within a range of 0.02-25.9% (FIG. 3). A skilled person in the art would be able to adopt appropriate values as the thresholds according to general principles of EM algorithm.

### Total Probability Module

In one embodiment, the present invention provides a total probability module which determines the total probability of an outcome of interest. As illustrated in FIG. 2, a plurality of conditional probabilities is input into the total probability module (22) for determining the probability of a particular aneuploidy such as a trisomy. In one embodiment, the conditional probabilities are produced or derived from data reported in literature, including but not limited to, probabilities of having trisomy based on the survival probabilities (i.e., spontaneous abortion “SA”, still birth “SB” and live birth “LB”), and survival probabilities based on maternal age “ma” and gestational week “gw”. In one embodiment, the present total probability module outputs the probability of trisomy including the probability of euploidy, the probability of maternal trisomy and the probability of paternal trisomy. The probabilities output by the total probability module can be described as “prior” probabilities since they are determined based on information and observation that are not derived from the fetus in question.

### Bayesian Module

In one embodiment, the present invention provides a Bayesian module which converts the likelihoods of various euploidy and aneuploidy status obtained in the previous modules into posterior probabilities which are the probabilities of the euploidy or aneuploidy given the observed genotypes. As illustrated in FIG. 2, the Bayesian module (23) derives the posterior probability for euploid, maternal trisomy and paternal trisomy based on the respective likelihoods and probabilities obtained from the EM algorithm module and the total probability module.

### Determining the Probability of Fetal Aneuploidy

This section illustrates a method for determining the probability of particular types of trisomy (T13, T18 and T21) using the present invention. A skilled person in the art would be able to apply the present invention to determine the probability of other types of fetal aneuploidy by selecting SNPs on chromosomes in question and using probability data relevant to the aneuploidy in question.

Fetal fraction is general indicator of the accuracy of the determination in the sense that the test results are more accurate if the fetal fraction has a higher value. Hence, thresholds for fetal fraction are established to evaluate the accuracy of a test result and shall be selected based on factors such as the method used for preparing the library, SNP selection, sequence quality and sequencing depth and the desired specificity. Provided that the fetal fraction is satisfactory, posterior probability of trisomy (i.e. the probability of trisomy with respect to the fetus in question) can be calculated based on the genotype/SNP data obtained from the sequencing data. In one embodiment of the present invention, the test achieves 99% specificity at a fetal fraction>2.1%.

When calculating the probability that the fetus carrying the observed genotype (i.e. at SNPs of interest obtained from sequencing) reflects a specific trisomy, the probability that a fetus from the general population has a specific trisomy (expressed as P(Dᵢ)) needs to first be calculated based on all its survival probabilities (i.e., spontaneous abortion “SA”, still birth “SB” and live birth “LB”) and that of the general population's. Formula (1), adapted from the Bayes rule, calculates the probability of a fetus having a specific trisomy, where other information including maternal age (expressed as ma) and gestational week (expressed as gw) are required.

P(Dᵢ)=P(Dᵢ|SA)P(SA|ma,gw)+P(Dᵢ|SB)P(SB|ma,gw)+P(Dᵢ|LB)P(LB|ma,gw) (1)

When P(Dᵢ) is input along with G, which represents the maternal-fetal genotype, P(Dᵢ|G) which is the likelihood that a fetus carrying the observed genotype of interest has a specific trisomy, can be obtained by formula (2). In one embodiment, whether the fetus has trisomy can be determined based on the genotype data obtained from sequencing.

P(Dᵢ|G) is calculated as the probability that people suffering trisomy Dᵢ carry the observed genotype of interest over the summation of the probability that people having different types of trisomy and people not having these trisomic conditions carry the observed genotype, i.e. P(G|Dᵢ)P(Dᵢ) divided by Σₖ P(G|Dₖ)P(Dₖ)+P(G|N)P(N), where Σₖ P(G|Dₖ)P(Dₖ)+P(G|N)P(N) represents the general probability of the observed fetal genotype in four scenarios (i.e. T13, T18, T21 and N where P(N)=1−Σₖ P(Dₖ)).

$$P ( {D}_{i} \vert G ) = \frac{p ( G \vert {D}_{i} ) P ( {D}_{i} )}{{\sum }_{k} P ( G \vert {D}_{k} ) P ( {D}_{k} ) + P ( G \vert N ) P ( N )} = \frac{\frac{P ( G \vert {D}_{i} )}{P ( G \vert N )} P ( {D}_{i} )}{{\sum }_{k} \frac{P ( G \vert {D}_{k} )}{P ( G \vert N )} P ( {D}_{k} ) + P ( N )} \qquad ( 2 )$$

General probability of the observed fetal genotype in other scenarios can also be obtained and used in a similar fashion as described above.

### Genotype Probability and the Expected Reference Allele Frequency of Each Combination of the Maternal-Fetal Genotype

Genotype probability would serve to determine the prior frequency (e.g. the probabilities described in Goya, R et al. for cancer-related genotypes [1]) of different maternal-fetal genotype combinations in maternal plasma. The present invention provides various values of expected reference allele frequencies as determined from a parameterp, which by definition, refers to the allele frequency of the reference allele. The value of p is pre-determined to achieve the desired sensitivity (e.g. 99% specificity). The expected reference allele frequency provided herein would then serve as a parameter for modeling the allelic counts at each SNP locus. The prior frequency and the expected reference allele frequency serve as inputs of an algorithm for calculating the likelihoods of each type of aneuploidy as well as for deriving the fetal fraction [3]. Likelihoods of different fetal aneuploidies given the derived fetal fraction are calculated using the algorithm described herein.

The present invention is able to analyze a vast number of SNPs without limitation. In one embodiment, the number of SNPs to be analyzed in a single analysis is adjusted according to the sequencing depth. For example, fewer SNPs can be analyzed if the sequencing depth obtained is higher.

For example, when fetal fraction (ff)=10%, euploid fetus should give possible allele frequencies of 0, 0.05, 0.45, 0.5, 0.55, 0.95 and 1.0 (Table 3). In contrast, maternal trisomy with 100% isodisomy (i.e., both copies of a chromosomal set being inherited from one parent only) should have possible allele frequencies of 0, 0.047619, 0.4285714, 0.47619, 0.52381, 0.571428, 0.95238 and 1.0. These two models give different likelihoods, and the ratio between the two likelihoods can be converted into a probability if the maternal age and gestational week are considered. Overall, the present algorithm takes into account a set of factors including maternal age and gestational week and allele frequencies of the two alleles of biallelic SNPs for different aneuploidy models, and translates likelihood ratios of these aneuploidy models into posterior probability of a particular aneuploidy.

Euploid: One Maternal Chromosome and One Paternal Chromosome Biological Mother

Let A be the reference allele and B be the alternate allele.

Let p be the allele frequency of the reference allele and 1-p be the allele frequency of the alternate allele.

TABLE 2
Probability of genotype based on reference allele frequency
for euploid when p = 0.6

| Mother genotype_{fetus genotype} | Probability of genotype | p = 0.6 |
|---|---|---|
| AA_{AA} | p³ | 0.2160 |
| AA_{AB} | p(1 − p)² | 0.0960 |
| AB_{AA} | p²(1 − p) | 0.1440 |
| AB_{AB} | p(1 − p) | 0.2400 |
| AB_{BB} | p(1 − p)² | 0.0960 |
| BB_{AB} | p²(1 − p) | 0.1440 |
| BB_{BB} | (1 − p)³ | 0.0640 |

Let f be the fetal fraction

TABLE 3
Fetal fraction for euploid when f = 0.1

| Mother | Expected reference | |
|---|---|---|
| genotype_{fetus genotype} | allele frequency | f = 0.1 |
| AA_{AA} | 1 | 1.0000 |
| AA_{AB} | $1 - \frac{f}{2}$ | 0.9500 |
| AB_{AA} | $\frac{1 + f}{2}$ | 0.5500 |
| AB_{AB} | 0.5 | 0.5000 |
| AB_{BB} | $\frac{1 - f}{2}$ | 0.4500 |
| BB_{AB} | $\frac{f}{2}$ | 0.0500 |
| BB_{BB} | 0 | 0.000 |

Maternal Trisomy: Two Maternal Chromosomes and One Paternal Chromosome

Let P₁ be the percentage of isodisomy.
Let A be the reference allele and B be the alternate allele.
Let p be the allele frequency of the reference allele and 1-p be the allele frequency of the alternate allele.

TABLE 4
Probability of genotype based on reference allele frequency for
maternal trisomy when p = 0.6

| Mother | | |
|---|---|---|
| genotype_{fetus genotype} | Probability of genotype | p = 0.6 |
| AA_{AAA} | P³ | 0.2160 |
| AA_{AAB} | p²(1 − p) | 0.1440 |
| AB_{AAA} | Pᵢp²(1 − p) | Pᵢ 0.1440 |
| AB_{AAB} | p(1 − p)(Pᵢ − 3Pᵢp + 2p) | 0.288-0.192Pᵢ |
| AB_{ABB} | p(1 − p)(3Pᵢp + 2 − 2Pᵢ − 2p) | 0.192-0.048Pᵢ |
| AB_{BBB} | Pᵢp(1 − p)² | Pᵢ 0.0960 |
| BB_{ABB} | p(1 − p)² | 0.0960 |
| BB_{BBB} | (1 − p)³ | 0.0640 |

Let f be the fetal fraction

TABLE 5
Fetal fraction for maternal trisomy when f = 0.1

| Mother | Expected reference | |
|---|---|---|
| genotype_{fetus genotype} | allele frequency | f = 0.1 |
| AA_{AAA} | 1 | 1.0000 |
| AA_{AAB} | $\frac{2}{2 + f}$ | 0.9524 |
| AB_{AAA} | $\frac{1 + 2 f}{2 + f}$ | 0.5710 |
| AB_{AAB} | $\frac{1 + f}{2 + f}$ | 0.5238 |
| AB_{ABB} | $\frac{1}{2 + f}$ | 0.4762 |
| AB_{BBB} | $\frac{1 - f}{2 + f}$ | 0.4290 |
| BB_{ABB} | $\frac{f}{2 + f}$ | 0.0476 |
| BB_{BBB} | 0 | 0.0000 |

Paternal Trisomy: One Maternal Chromosomes and Two Paternal Chromosomes

Let P₁ be the percentage of isodisomy.
Let A be the reference allele and B be the alternate allele.
Let p be the allele frequency of the reference allele and 1-p be the allele frequency of the alternate allele.

TABLE 6
Probability of genotype based on reference allele frequency for paternal
trisomy when p = 0.6

| Mother genotype_{fetus genotype} | Probability of genotype | p = 0.6 |
|---|---|---|
| AA_{AAA} | p³(Pᵢ + p − Pᵢp) | Pᵢ 0.2160 + (1 − Pᵢ) 0.1296 |
| AA_{AAB} | 2p³(1 − p)(1 − Pᵢ) | (1 − Pᵢ) 0.1728 |
| AA_{ABB} | p²(1 − p)(1 − p + Pᵢp) | Pᵢ 0.1440 + (1 − Pᵢ) 0.0576 |
| AB_{AAA} | p²(1 − p)(Pᵢ + p (1 − Pᵢ)) | Pᵢ 0.1440 + (1 − Pᵢ) 0.0864 |
| AB_{AAB} | p²(1 − p)(Pᵢ + (1 − Pᵢ)(2 − | Pᵢ 0.1440 + (1 − Pᵢ) 0.2016 |
| | p)) | |
| AB_{ABB} | p(1 − p)²(Pᵢ + (1 − Pᵢ)(1 + | Pᵢ 0.0960 + (1 − Pᵢ) 0.1536 |
| | p)) | |
| AB_{BBB} | p(1 − p)²(Pᵢ + (1 − Pᵢ)(1 − | Pᵢ 0.0960 + (1 − Pᵢ) 0.0384 |
| | p)) | |
| BB_{AAB} | p(1 − p)²(p − Pᵢp + Pᵢ) | Pᵢ 0.0960 + (1 − Pᵢ) 0.0576 |
| BB_{ABB} | 2p(1 − p)³(1 − Pᵢ) | (1 − Pᵢ) 0.0768 |
| BB_{BBB} | (1 − p)³(1 − p + Pᵢp) | Pᵢ 0.0640 + (1 − Pᵢ) 0.0256 |

Let f be the fetal fraction

TABLE 7
Fetal fraction for paternal trisomy when f = 0.1

| Mother | Expected reference | |
|---|---|---|
| genotype_{fetus genotype} | allele frequency | f = 0.1 |
| AA_{AAA} | 1 | 1.0000 |
| AA_{AAB} | $\frac{2}{2 + f}$ | 0.9524 |
| AA_{ABB} | $\frac{2 - f}{2 + f}$ | 0.9048 |
| AB_{AAA} | $\frac{1 + 2 f}{2 + f}$ | 0.5710 |
| AB_{AAB} | $\frac{1 + f}{2 + f}$ | 0.5238 |
| AB_{ABB} | $\frac{1}{2 + f}$ | 0.4762 |
| AB_{BBB} | $\frac{1 - f}{2 + f}$ | 0.4290 |
| BB_{AAB} | $\frac{2 f}{2 + f}$ | 0.0952 |
| BB_{ABB} | $\frac{f}{2 + f}$ | 0.0476 |
| BB_{BBB} | 0 | 0.0000 |

Ratios Between Maternal and Paternal Trisomies

Numbers for the five possible causes of trisomy are taken from literature [4]:

TABLE 8
Ratios of trisomies from different causes

| Causes of Trisomy | Number (Total: 642) | Ratios |
|---|---|---|
| Maternal Meiosis I | 420 | 0.6542 |
| Maternal Meiosis II | 150 | 0.2336 |
| Paternal Meiosis I | 22 | 0.0342 |
| Paternal Meiosis II | 30 | 0.0467 |
| Mitosis | 20 | 0.0311 |

Assuming the parental ratios of T13, T18 and T21 are the same and half of Mitosis are maternal and half are paternal, we have the following:

TABLE 9
Ratios between maternal and paternal trisomies

| Type of trisomy | Type of meiosis | Number | Ratios |
|---|---|---|---|
| Maternal trisomy | Maternal meiosis I, | 580 | 0.9034 |
| | Maternal meiosis II, | | |
| | Mitosis/2 | | |
| Paternal trisomy | Paternal meiosis I, | 62 | 0.0966 |
| | Paternal meiosis II, | | |
| | Mitosis/2 | | |

Posterior Distribution from the Likelihood

The posterior probability of the fetus having a particular trisomy given each genotype is derived. The source of the data and the calculations for deriving the posterior probability is presented. An example demonstrates how to calculate the posterior probability.

$$P ( {D}_{i} \vert G ) = \frac{P ( G \vert {D}_{i} ) P ( {D}_{i} )}{{\sum }_{k} P ( G \vert {D}_{k} ) P ( {D}_{k} ) + P ( G \vert N ) P ( N )} = \frac{\frac{P ( G \vert {D}_{i} )}{P ( G \vert N )} P ( {D}_{i} )}{{\sum }_{k} \frac{P ( G \vert {D}_{k} )}{P ( G \vert N )} P ( {D}_{k} ) + P ( N )} \qquad ( 2 )$$

where P(G|Dᵢ) is the likelihood of a person with the genotype G having the condition Dᵢ, P(Dᵢ) is the prior probability of the condition, N represents cases not carrying the tested conditions, Dᵢ represents different types of tested ploidy (maternal trisomy, paternal trisomy, etc.).

The model calculating the probability of trisomy can be extended into triploidy.

### Source of the Data

P(Dᵢ), P(N): for trisomy, we derive P(Dᵢ) by calculation. Let P(N)=1−P(Dᵢ).
P(G|D), P(G|N): calculated from the program that is an extension of SNVMix2 [1].

### Getting P(SA|ma,gw)

Data are downloaded from https://datayze.com/miscarriage-chart.php and three data points of P(SA|ma,gw) at maternal age <35, 35-39 and 40 are obtained (Table 10). To improve the accuracy of P(LB|ma,gw), quadratic regressions across all gestational ages and at maternal age 25, 37 and 44 are performed. This is done in R (a programming language for statisticians).

TABLE 10
Regression data of P(SA|ma, gw) at different
gestational week and maternal age.

| Ges- | | | | | | |
|---|---|---|---|---|---|---|
| tational | Maternal age | | | Quadratic regression | | |
| week | <35 | 35-39 | ≥40 | intercept | parameter1 | parameter2 |
| 3 | 0.282 | 0.39 | 0.738 | 1.179013 | −0.18213 | 0.003373 |
| 4 | 0.235 | 0.325 | 0.616 | 1.007991 | −0.18289 | 0.003386 |
| 5 | 0.179 | 0.247 | 0.468 | 0.747151 | −0.18352 | 0.003393 |
| 6 | 0.127 | 0.176 | 0.333 | 0.367697 | −0.18133 | 0.003363 |
| 7 | 0.082 | 0.114 | 0.215 | −0.1117 | −0.1787 | 0.003325 |
| 8 | 0.049 | 0.068 | 0.129 | −0.5748 | −0.18207 | 0.003377 |
| 9 | 0.033 | 0.046 | 0.086 | −1.09895 | −0.17369 | 0.003248 |
| 10 | 0.023 | 0.032 | 0.061 | −1.3132 | −0.18342 | 0.003402 |
| 11 | 0.019 | 0.027 | 0.05 | −1.46927 | −0.18063 | 0.003317 |
| 12 | 0.016 | 0.022 | 0.041 | −1.76096 | −0.17707 | 0.003284 |
| 13 | 0.012 | 0.017 | 0.033 | −1.94844 | −0.18546 | 0.00346 |
| 14 | 0.01 | 0.013 | 0.025 | −1.66819 | −0.21163 | 0.003766 |
| 15 | 0.007 | 0.01 | 0.019 | −2.68793 | −0.1725 | 0.003262 |
| 16 | 0.005 | 0.007 | 0.013 | −3.05903 | −0.16904 | 0.003179 |
| 17 | 0.004 | 0.005 | 0.01 | −2.07088 | −0.24385 | 0.004233 |
| 18 | 0.003 | 0.004 | 0.007 | −3.68355 | −0.15867 | 0.002946 |
| 19 | 0.001 | 0.002 | 0.004 | −6.34316 | −0.07687 | 0.002172 |

### Getting P(SB|ma,gw) and P(LB|ma,gw)

P(SB|age,week)=P(SB|age)

The values of P(SB|age) are taken from the literature [5].

The data from the literature is as follows:

TABLE 11
Probability of stillbirth

| Maternal | Total number of | Number of | Probability of |
|---|---|---|---|
| Age | samples | stillbirth | stillbirth |
| <20 | 6463 | 87 | 0.0135 |
| 24 | 89373 | 639 | 0.0071 |
| 29 | 125138 | 703 | 0.0056 |
| 34 | 52245 | 383 | 0.0073 |
| ≥35 | 18087 | 260 | 0.0144 |

A regression on the data with a third-degree polynomial with age 17, 22, 27, 32, 40 is performed.

The formula is 0.08243−0.006765×age+0.0001834×(age)²−0.00000142×(age)³

$$P ( LB \vert age , week ) = 1 - P ( SA \vert age , week ) - P ( SB \vert age , week ) = 1 - P ( SA \vert age , week ) - P ( SB \vert age )$$

The values P(LB|age,week) are therefore as follows:

TABLE 12
Value of P(LB|age, week)

| Gestational | Age | | | | | |
|---|---|---|---|---|---|---|
| week | <20 | 24 | 29 | 34 | 35-39 | ≥40 |
| 3 | 0.7045 | 0.7109 | 0.7124 | 0.7107 | 0.5956 | 0.2476 |
| 4 | 0.7515 | 0.7579 | 0.7594 | 0.7577 | 0.6606 | 0.3696 |
| 5 | 0.8075 | 0.8139 | 0.8154 | 0.8137 | 0.7386 | 0.5176 |
| 6 | 0.8595 | 0.8659 | 0.8674 | 0.8657 | 0.8096 | 0.6526 |
| 7 | 0.9045 | 0.9109 | 0.9124 | 0.9107 | 0.8716 | 0.7706 |
| 8 | 0.9375 | 0.9439 | 0.9454 | 0.9437 | 0.9176 | 0.8566 |
| 9 | 0.9535 | 0.9599 | 0.9614 | 0.9597 | 0.9396 | 0.8996 |
| 10 | 0.9635 | 0.9699 | 0.9714 | 0.9697 | 0.9536 | 0.9246 |
| 11 | 0.9675 | 0.9739 | 0.9754 | 0.9737 | 0.9586 | 0.9356 |
| 12 | 0.9705 | 0.9769 | 0.9784 | 0.9767 | 0.9636 | 0.9446 |
| 13 | 0.9745 | 0.9809 | 0.9824 | 0.9807 | 0.9686 | 0.9526 |
| 14 | 0.9765 | 0.9829 | 0.9844 | 0.9827 | 0.9726 | 0.9606 |
| 15 | 0.9795 | 0.9859 | 0.9874 | 0.9857 | 0.9756 | 0.9666 |
| 16 | 0.9815 | 0.9879 | 0.9894 | 0.9877 | 0.9786 | 0.9726 |
| 17 | 0.9825 | 0.9889 | 0.9904 | 0.9887 | 0.9806 | 0.9756 |
| 18 | 0.9835 | 0.9899 | 0.9914 | 0.9897 | 0.9816 | 0.9786 |
| 19 | 0.9855 | 0.9919 | 0.9934 | 0.9917 | 0.9836 | 0.9816 |

### Calculating P(Dᵢ)

To calculate P(Dᵢ), the following formula is applied:

P(Dᵢ)=P(Dᵢ|SA)P(SA|ma,gw)+P(Dᵢ|SB)P(SB|ma,gw)+P(Dᵢ|LB)P(LB|ma,gw) (1)

where ma is the maternal age and gw is gestational week.

### Conditional Probability

In this analysis, the parameters taken from the literature [6-9] are applied.

TABLE 13
Conditional probabilities for various trisomy based on
survival probabilities

| | | | Prob- | | |
|---|---|---|---|---|---|
| | Probability | | ability | | Probability |
| P(T21\|SA) | 0.0319 | P(T21\|SB) | 0.0921 | P(T21\|LB) | 0.0005 |
| P(T13\|SA) | 0.0319 | P(T13\|SB) | 0.0026 | P(T13\|LB) | 0.0000 |
| P(T18\|SA) | 0.0160 | P(T18\|SB) | 0.0102 | P(T18\|LB) | 0.0042 |

### Example

Let Di to be trisomy T21,

| | Values |
|---|---|
| Parameters | |
| P(T21\|SA) | 3.19% |
| P(SA\|ma, gw) | 15% |
| P(T21\|SB) | 9.21% |
| P(SB\|ma, gw) = P(SB\|ma) | 15% |
| P(T21\|LB) | 0.05% |
| Likelihood | |
| Non-trisomy 21 fetus | e⁻²⁰⁶²²¹ |
| Maternal trisomy 21 | e⁻²⁰⁶²⁰⁹ |
| Paternal trisomy 21 | e⁻²⁰⁶²⁷¹ |

$$P ( T 21 \vert ma , gw ) = P ( T 21 \vert SA ) P ( SA \vert ma , gw ) + P ( T 21 \vert SB ) P ( SB \vert ma , gw ) + P ( T 21 \vert LB ) P ( SB \vert ma , gw ) = P ( T 21 \vert SA ) P ( SA \vert ma , gw ) + P ( T 21 \vert SB ) P ( SB \vert ma ) + P ( T 21 \vert LB ) ( 1 - P ( SB \vert ma , gw ) - P ( SB \vert ma ) )$$

By substituting the values, P(T21)=0.01895.

According to the equation for P(Dᵢ|G),

$$P ( maternal trisomy \vert genotype ) = \frac{\frac{{e}^{- 206209}}{{e}^{- 206221}} \times 0.01895 \times 0.9034}{\begin{matrix}( 1 - 0.01895 ) + \frac{{e}^{- 206209}}{{e}^{- 206221}} \times 0.01895 \times 0.9034 + \\ \frac{{e}^{- 206271}}{{e}^{- 206221}} \times 0.01895 \times 0.0966\end{matrix}}$$

The ratios of trisomies (i.e., 0.9034 and 0.0966) are provided according to Table 9 in this document. The value 0.01895 is the probability of T21.

In one embodiment, the method determines whether the fetus in question has aneuploidy by comparing the determined probability of aneuploidy and a cutoff value which produces a pre-determined sensitivity. In one embodiment, a cut-off value of 90% gives rise to a sensitivity of over 99%.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### EXAMPLES

### Example 1

### Sample Preparation—Purification of Cell-Free DNA from Maternal Blood or Plasma Sample

This example illustrates one embodiment of extraction of cell-free DNA from maternal whole blood or plasma sample using Promega Maxwell® Rapid Sample Concentrator (RSC) and determination of the concentration of the extracted genomic DNA using Qubit® Fluorometer.

10 mL whole blood sample was collected from a pregnant subject and stored in cfDNA blood tube. The sample was then processed according to the following protocols:

Preparing Plasma
1. Centrifuge the whole blood from cfDNA blood tubes at 3000 rpm for 5 min.
2. Aliquot out all the plasma and centrifuge the collected plasma at 14000 rpm for 10 min. 3. Collect and store the supernatant in a new 2 mL screw cap tube at 4° C. until further use, or −20° C. for long-term storage.

Binding of Circulating Nucleic Acid to Magnetic Resin
1. Add 2 mL of binding buffer to a 50 mL falcon tube.

2. Add 140 μL of magnetic resin to the falcon tube.
3. Add 2 mL of plasma sample to the falcon tube (ratio of sample to binding buffer is 1:1).
4. Mix on roller shaker for 45 min.
5. Centrifuge the 50 mL falcon at 6000 rcf for 3 min to pellet the resin (Eppendorf 5430/R) or 4000 rpm for 2 min (Eppendorf 5810/R).
6. Remove the supernatant carefully by a P1000 pipette and avoid disturbing the beads pellet.

Preparation of Maxwell Cartridges
1. Briefly swing down the cartridge to bring down all the volume.
2. Place the cartridge in Maxwell Deck Tray with well #1 furthest away from the elution tube.
3. Snap the cartridge into position until a “CLICK” sound is heard.
4. Peel the seal from the operator's direction to the largest well.
5. Place an elution tube into the elution tube position for each cartridge in the tray.
6. Add 75 μL of elution buffer to the bottom of elution tube.
7. Place a plunger into the well #8 of each cartridge.
8. Re-suspend the magnetic resin in the 50 mL falcon with the buffer from well 1 (the large
well) of the cartridge by using a P1000 pipette.
9. Transfer the resin and the liquid back to well 1 of the Maxwell cartridge.
10. Place the tray back into the instrument.

Instrument Run
1. Turn on Maxwell Instrument and the Tablet computer.

2. Select Start on Home screen.
3. Select extraction method: “LV ccf DNA Custom”.
4. Select cartridge positions to be run and name the sample.
5. Verify: samples were added to well #1 of cartridges, cartridges were loaded into the correct position, elution tube contained the correct amount of elution buffer, plunger placed into well #8, and the elution tube lid remained open.
6. Click start to close the instrument door and start processing.
7. Stored purified cell-free DNA at −30° C.

Determination of concentration of cell-free DNA purified using Qubit® dsDNA High Sensitivity assay kit and Qubit® instrument:

Purified cell-free DNA obtained from the previous step was vortexed and spun down using benchtop centrifuge. 2 μL cell-free DNA solution was taken and the concentration of cell-free DNA was measured according to the following protocols:

Reaction Mixture Preparation
1. Prepare reaction mixture according the following ratio: 199 μL of reaction buffer to 1 μL of florescent dye.
2. Mix thoroughly.
3. Aliquot 190 μL of the reaction mixture into two Qubit reaction tube to set up two standards.
4. Add 10 μL of Standard 1 solution and Standard 2 solution respectively to the two standard tubes, each of which contains 190 μL, of reaction mixture. Mix thoroughly.
5. Aliquot 198 μL of the resulting reaction mixture into Qubit reaction tube for measuring the cell-free DNA concentration.
6. Add 2 μL of cell-free DNA into the reaction tube which contains 198 μL, of reaction mixture.
7. Mix the mixtures in each standard and sample reaction tubes thoroughly and then bring the mixtures down by centrifuging the reaction tubes.

Instrument Run
1. Turn on Qubit® instrument.

2. Select dsDNA on home screen.
3. Select dsDNA: High sensitivity assay on home screen.
4. Select Read Standards on home screen.
5. Insert Standard 1 tube into sample chamber and press Read standard.
6. Insert Standard 2 tube into sample chamber and press Read standard.
7. After the Fluorescence vs. Concentration graph show on home screen.
8. Insert sample tube into sample chamber and press Run samples.
9. Choose input sample volume and output sample units, press Read tube.

### Example 2

### Library Preparation for Target Enrichment—Protocols for Hybridization-Based Approach

In the present invention, hybridization-based library preparation builds on the SeqCap manufacturer's protocol. In the following protocols and procedures, slight differences from the manufacturer's version are introduced to customize and optimize the workflow so as to achieve better sequence readings in the later stages.

The procedures comprise fragmentation, end repair and A-tailing of DNA, adapter ligation, library amplification (i.e., pre-enrichment amplification), post-amplification cleanup, sample hybridization with SeqCap Probe Pool (i.e., target enrichment) and post-hybridization amplification (i.e., post-enrichment amplification). Detailed protocols are as follows:

Optimized SeqCap EZ Workflow

### A. Put the AMPure XP Beads at Room Temperature

B. Prepare DNA (1 ng-1 μg Recommended for Cell-Free DNA)
1. Maximize volume-50 W plasma Input

### C. End Repair and A-Tailing

Kit Used: KAPA HyperPrep Kit
1. Prepare the following Master Mix (RT) for each sample

| Component | Volume (μl) |
|---|---|
| ccfDNA | 50 |
| End Repair & A-Tailing Buffer | 7 |
| End Repair & A-Tailing Enzyme Mix | 3 |
| Total volume | 60 |

2. Vortex gently and spin down
3. Start the following program in Thermocycler

| Step | Temp | Time |
|---|---|---|
| End Repairing & A-Tailing | 20° C. | 30 min |
| | 65° C. | 30 min |
| HOLD | 4° C. | ∞ |

4. Proceed Immediately to Adapter Ligation

### D. Adapter Ligation

1. Prepare the following Master Mix (for 1 sample)

| Component | Volume (μl) |
|---|---|
| PCR-grade water | 5 |
| Ligation Buffer | 30 |
| DNA Ligase | 10 |
| Total: | 50 |

2. Add the master mix to each sample (60 μl).
3. Add 5 μl undiluted SeqCap Indexed Adapter to each sample
   (Note: Different sample uses different index adapter)
4. Mix thoroughly and spin down
5. Incubate at 20° C. in thermocycler for 15 min
6. Proceed to Post-ligation Cleanup

### E. Post-Ligation Cleanup

1. In the same tube, perform cleanup by combing the following components.

| Component | Volume (μl) |
|---|---|
| Adapter ligation reaction product | 110 |
| AMPure XP Reagent | 88 |
| Total: | 198 |

2. Record the index used for each sample.
3. Mix it thoroughly by pipetting up and down. (not vortex)
4. Incubate at Room Temperature for 5 min to bind DNA to the beads.
5. Place the tube on a magnet to capture the beads. Incubate until the liquid is clear. (>=5 min)
6. Carefully remove and discard the supernatant.
7. Keeping the tube on the magnet, add 200 μl freshly-made 80% ethanol.
8. Incubate the tube on the magnet at room temperature for >=30 s
9. Carefully remove and discard the ethanol.
10. Keeping the tube on the magnet, add 200 μl freshly-made 80% ethanol.
11. Incubate the tube on the magnet at room temperature for >=30 s
12. Carefully remove and discard the ethanol. Try to remove all residual ethanol without disturbing the beads.
13. Dry the beads at room temperature for 4-5 min.
   (Note: Over-drying the bead will reduce the yield)
14. Remove the tube from the magnet.
15. Thoroughly re-suspend the beads in 23 μl of elution buffer.
16. Incubate the tube at room temperature for 2 min to elute DNA off the beads.
17. Place the tube on a magnet to capture the bead. Incubate until the liquid is clear.
18. Transfer the clear 22 μl supernatant to 96-well plate to perform Library Amplification.

### F. Library Amplification

1. Assemble each library amplification reaction in 96-well plate as follow.

| Component | Volume (μl) |
|---|---|
| KAPA HiFi HotStart readyMix(2x) | 25 |
| KAPA Library Amplification Primer Mix(10x) | 5 |
| Adapter-ligated library | 20-22 |
| Total: | 50 |

2. Mix it thoroughly and spin down.
3. Amplify using the following steps (˜32 min)
   (Volume=50, heated lid=105° C., Ramp=3° C.)

| Step | Temp | Duration | Cycles |
|---|---|---|---|
| Initial Denaturation | 98° C. | 45 s | 1 |
| Denaturation | 98° C. | 15 | 7-8 |
| Annealing | 60° C. | 30 | |
| Extension | 72° C. | 30 | |
| Final extension | 72° C. | 1 min | 1 |
| Hold | 4° C. | ∞ | 1 |

4. Proceed directly to Post-amplification cleanup

### G. Post Amplification Cleanup

Prepare the following aliquots:
i. 80% ethanol
ii. AMPure XP reagent
iii. PCR-grade water
iv. Qubit Reagents—two standards and reagents for sample
1. Allow the AMPure XP beads to equilibrate to room temperature at least 30 min before use.
2. Vortex the AMPure XP beads to a homogenous state before use.
3. Add 90 μl AMPure XP beads to the 50 μl amplified sample library in a 96 well plate.
4. Mix it thoroughly by pipette up and down
5. Incubate at room temperature for 5 min to allow the DNA bind to the beads
6. Place the sample in a magnet collector. Incubate until the liquid is clear.
7. Remove and discard the supernatant without disturbing the beads.
8. Keep the sample on the magnet. Add 200 μl freshly-made 80% ethanol
9. Incubate at room temperature >=30 s
10. Remove and discard the ethanol
11. Keeping the sample on the magnet, add 200 μl freshly-made 80% ethanol
12. Incubate at room temperature >=30 s
13. Remove and discard the ethanol. Try to remove all residual ethanol without disturbing the beads.
14. Allow the beads to dry at room temperature for 4-5 min, NOT >5 min. Over drying can result in low yield.
15. Remove the samples from the magnetic collector.
16. Resuspend the DNA using 53 μl elution buffer.
17. Pipette up and down to mix the sample. Ensure all the beads are resuspended.
18. Incubate at room temperature for 2 min
19. Place the sample on the magnetic collector until the liquid is clean
20. Transfer 52 μl of supernatant to a new 1.5 ml tube.
21. Pipette 1.5 μl sample to another new 1.5 ml for Bioanalyzer in the next day.
22. Add 2 μl sample to 198 ul Qubit working solution for Qubit quantitation.
23. Store the entire sample at −20° C. before Hybridization.

### H. Hybridize the Sample and SeqCap EZ Probe Pool

Kits Used:
SeqCap HE Universal, SeqCap HE Index Oligos
COT Human DNA in SeqCap EZ Accessory Kit
Hybridization and Wash kit
1. Thaw the SeqCap HE Universal, SeqCap HE Index Oligos that match a DNA adapter index included in library pool
2. Thaw a sample library for pooling
3. Mix together equal amount of each amplified DNA sample libraries to obtain a single pool with a combined mass of 1 (eg. 0.25 μg for each 4 libraries to a single pool.) Total oligos=2000 pmol (2 μl of 1000 μM)
4. Prepare the Multiplex Hybridization Enhancing Oligo Pool
   i. SeqCap HE Universal Oligo=1000 pmol (1 μl of 1000 μM)
   ii. Mixture of HE Index oligos=1000 pmol
      (For example: HE Index oligos mixture=250 pmol (0.25 μl of 1000 μM) of each 4 HE Index oligos)
5. Prepare the Hybridization Mix according to the following table

| Component | Amount | | Volume (μl) |
|---|---|---|---|
| COT Human DNA | 5 | μg | 5 |
| Multiplex DNA sample Library Pool | 1 | μg | x |
| SeqCap HE Universal Oligo | 1000 | pmol | 1 |
| SeqCap HE Index 13 Oligo | 250 | pmol | 0.25 |
| SeqCap HE Index 14 Oligo | 250 | pmol | 0.25 |
| SeqCap HE Index 15 Oligo | 250 | pmol | 0.25 |
| SeqCap HE Index 18 Oligo | 250 | pmol | 0.25 |
| Total | | | 7 + x |

6. Determine the total volume of Hybridization Mix.
7. Add 2 volume of AMPure XP reagent to Hybridization Mix.
   (Eg. If the total volume of Hybridization mix=57 ul, then add 114 μl AMPure XP reagents to the mix.
8. Incubate the sample at room temperature for 10 min to allow the sample library to bind to the beads.
9. Place the sample on the magnetic collector to capture the beads until the liquid is clear.
10. Remove and discard the supernatant without disturbing the beads.
11. Keep the sample on the magnet. Add 190 μl freshly-made 80% ethanol
12. Incubate at room temperature >=30 s
13. Remove and discard the ethanol. Try to remove all residual ethanol without disturbing the beads.
14. Allow the beads to dry at room temperature for 4-5 min, NOT >5 min. Over drying can result in low yield.
15. Add the following reagents (volume for 1 sample pool) to resuspend the beads.
   i. 7.5 μl of 2× Hybridization Buffer (vial 5)
   ii. 3 μl of Hybridization Component A (vial 6)
16. Remove the sample from the magnetic collector and mix it thoroughly by pipetting up and down.
17. Incubate at room temperature for 2 min
18. Place sample on a magnetic collector until the liquid is clear.
19. Transfer 10.5 μl supernatant in a NEW PCR plate containing 4.5 μl of the SeqCap EZ probe pool.
20. Mix it thoroughly.
21. Perform hybridization incubation in the thermocycler using the following program. (Volume=15 μl, heated lid=57° C.)
   i. 95° C. for 5 min
   ii. 47° C. for 16-20 hrs
      Note: During the hybridization, it is important that the heated lid of the thermocycler is 10° C. above the hybridization temperature. The sample is important to maintain 47 until it is transferred to the capture beads.
22. Proceed the Wash and Recovery steps immediately after hybridization. DO NOT store the sample prior to wash and recovery.
23. Prepare Bead Wash buffer for next day use.

Next Day

### I. Wash and Recovery of Captured Multiplex Sample

Kits Used:
Hybridization and Wash kit
SeqCap Pure Capture Bead Kit
1. Prepare Bead Wash buffer as below

| | | Volume | |
|---|---|---|---|
| | Volume | of H₂O | Total Volume of 1X |
| Buffer Stock | add (μl) | (μl) | working buffer(μl) |
| 10X Stringent Wash Buffer | 44 | 396 | 400 |
| (vial 4) | | | |
| 10X Wash Buffer I (vial 1) | 33 | 297 | 300 |
| 10X Wash Buffer II (vial 2) | 22 | 198 | 200 |
| 10X Wash Buffer III (vial 3) | 22 | 198 | 200 |
| 2.5X Bead Wash Buffer (vial 7) | 220 | 330 | 500 |

Working buffer can be stored at room temperature up to 2 weeks. The volumes are calculated for 1 sample including 10% access volume. Scale it up if more than one sample used

2. Allow the capture beads containing in the SeqCap Pure Capture Bead Kit to equilibrate to room temperature for 30 min prior use.
3. Vortex the capture beads for 15 s before use to ensure a homogeneous mixture of beads.
4. Aliquot 50 μl of beads for each sample in a 1.5 ml tube.
5. Place the tube on magnetic collector until the liquid is clear.
6. Remove and discard the supernatant without disturbing the beads
7. Keep the tube on magnetic collector, add twice initial volume of beads of 1× Bead Wash Buffer (ie. 100 μl of Bead Wash buffer for 50 μl capture beads)
8. Remove tube from the magnetic collector and mix it thoroughly by pipetting up and down
9. Place the tube on the magnetic collector until the liquid is clear
10. Remove and discard supernatant
11. Repeat Step 7-10 (total: 2 washes)
12. Add 1× the initial volume of beads of 1× Bead Wash Buffer (ie. 50 μl per capture)
13. Remove the tube from magnetic collector and mix it thoroughly by pipetting up and down
14. Aliquot 50 μl of resuspended beads into a NEW PCR plate for each capture.
15. Place the tube on magnetic collector until the liquid is clear.
16. Remove and discard the supernatant without disturbing the beads
17. The beads are now ready to bind the captured DNA. Proceed immediately to the next step. (DO not allow the capture beads to dry out)

### J. Bind DNA to the Capture Bead and Washing Step

1. Transfer 10.5 μl hybridization sample to the PCR plate containing the Capture beads from previous step.
2. Mix it thoroughly by pipetting up and down
3. Bing the capture DNA to beads by incubate the sample in the thermocycler at 47° C. for 15 min (headed lid set to 57° C.)
4. Remove the sample from thermocycler. Keep the thermocycler at 47° C. with heated lid at 57° C. for the following step.
5. Add 100 μl of 1× Wash Buffer Ito the 15 μl of Capture Bead Mix.
6. Mix thoroughly.
7. Place the tube on magnetic collector until the liquid is clear.
8. Remove and discard the supernatant without disturbing the beads
9. Add 200 μl of 1× Stringent Wash Buffer
10. Remove the tube from magnetic collector and mix it thoroughly by pipetting up and down
11. Place on the thermocycler per-heated to 47° C., close lid (set to 57° C.) and incubate for 5 min.
12. After incubation, remove the sample from thermocycler and place the tube on magnetic collector until the liquid is clear.
13. Remove and discard the supernatant without disturbing the beads
14. Repeat Step 9-13. (Total 2 washes using Stringent Wash buffer)
15. Add 200 μl×Wash Buffer I.
16. Mix it thoroughly by pipetting up and down to ensure the mixture is homogeneous.
17. Incubate at room temperature for 1 min
18. Place the tube on magnetic collector until the liquid is clear.
19. Remove and discard the supernatant without disturbing the beads
20. Add 200 μl×Wash Buffer II
21. Mix it thoroughly by pipetting up and down to ensure the mixture is homogeneous.
22. Incubate at room temperature for 1 min
23. Place the tube on magnetic collector until the liquid is clear.
24. Remove and discard the supernatant without disturbing the beads
25. Add 200 μl×Wash Buffer III
26. Mix it thoroughly by pipetting up and down to ensure the mixture is homogeneous.
27. Incubate at room temperature for 1 min
28. Place the tube on magnetic collector until the liquid is clear.
29. Remove and discard the supernatant without disturbing the beads
30. Remove the sample from magnetic collector.
31. Add 20 μl PCR-grade water to the plate well containing bead-bound DNA sample.

The beads plus captured DNA will be used as template in the LM-PCR.

### K. Amplify Captured Multiplex DNA Sample Using LM-PCR

Kit Use: SeqCap EZ Accessory Kit v2
1. Resuspend the Post-LM-PCR Oligos
2. Prepare a Master Mix of the following reagents

| Post-Capture LM-PCR Master Mix | Per Individual PCR reaction (μl) |
|---|---|
| KAPA HiFi HotStart readyMix (2X) | 25 |
| Post-LM-PCR Oligos 1 &2, 5 μM | 5 |
| Total | 30 |

The post-capture LM-PCR oligos and the KAPA HiFi HotStart ReadyMix (2X) are in SeqCap EZ Accessory Kit v2.

3. Keep the sample in the plate, add 30 μl Post-Capture LM-PCR Master Mix.
4. Mix thoroughly by pipetting up and down. Do not spin.
5. Perform the Post-Capture PCR Amplification in the thermocycler using the following program. (Set the ramp=3° C.)˜-20 min

Check the Cycle No. Before Run

| | | | |
|---|---|---|---|
| Step 1 | 98° C. | 45 s | 1 cycle |
| Step 2 | 98° C. | 15 s | 14 cycles |
| Step 3 | 60° C. | 30 s | |
| Step 4 | 72° C. | 30 s | |
| Step 6 | 72° C. | 1 min | 1 cycle |
| Step 7 | 4° C. | Hold | |

6. Proceed to the cleanup step

### L. Purification of the Amplified Captured Multiplex DNA Sample Using AMPure XP Beads

1. Make sure the AMPure XP beads is at room temperature at least 30 min prior use.
2. Vortex the beads for 10 s to ensure it is a homogeneous state
3. Add 90 μl AMPure beads to the 150 μl amplified captured multiplex DNA sample library in a 96 well plate.
4. Mix it thoroughly by pipetting up and down.
5. Incubate at room temperature for 5 min for sample bind to the beads
6. Place the tube on magnetic collector until the liquid is clear.
7. Remove and discard the supernatant without disturbing the beads
8. Keep the sample on magnetic collector. Add 200 μl freshly-made 80% ethanol
9. Incubate at room temperature >=30 s
10. Remove and discard the ethanol
11. Keep the sample on magnetic collector. Add 200 μl freshly-made 80% ethanol
12. Incubate at room temperature >=30 s
13. Remove and discard the ethanol. Try to remove all residual ethanol without disturbing the beads
14. Allow the beads to dry at room temperature for 4-5 min. (Not >5 min)
15. Remove the sample from the magnetic collector
16. Resuspend the DNA sample using 53 μl elution buffer (EA buffer)
17. Pipette up and down several times to ensure that all the beads are resuspended
18. Incubate at room temperature for 2 min
19. Place the tube on magnetic collector until the liquid is clear.
20. Transfer 53 μl supernatant in a NEW 1.5 ml tube
21. Measure the sample concentration by using Qubit and sample quality by Bioanalyzer

### M. Calculate the Library Input to Sequencer

[Quantity (ng/μl)/(660 g/mol×average library size in library)]×10\hat{ }6 =10 nM or 4 nM

Average library size in library=300 bp

### Example 3

### Library Preparation for Target Enrichment—Protocols for Amplicon Based Approach

Modifications have been made to the QIAseq Library Preparation protocols so as to achieve the desired result. Detailed protocols are as follows:
Determine the amount of input it and control the DNA to 10-40 ng
Reaction clean up: Clean the plates with QIAseq Beads
Centrifuge DNA sample by briefly centrifuge it and then mix it by pipetting up and 7-8 times and then centrifuge again.

Fragmentation, End-Repairing and A-Tailing
1. Prepare reaction mixture.

| Component | Volume/reaction |
|---|---|
| DNA | variable |
| Fragmentation buffer, 10x | 2.5 μl |
| FERA solution | 0.75 μl |
| Nuclease-free water | variable |
| Total | 20 μl |

2. Perform DNA fragmentation, end-repair and A-addition by adding 50 μL Fragmentation Enzyme mix to each reaction. Briefly centrifuge, mix by pipetting up and down 7-8 times and briefly centrifuge again.
3. Pre-chill thermal cycler at 4 degrees Celsius. Incubation time refer to the below table.
4. Transfer tubes to pre-chilled thermal cycler and resume cycling programme.
5. Allow thermal cycler to return to 4 degrees Celsius.

| Step | Incubation temperature | Incubation time |
|---|---|---|
| 1 | 4° C. | 1 min |
| 2 | 32° C. | 14 min |
| 3 | 72° C. | 30 min |
| 4 | 4° C. | Hold |

Adapter Ligation
1. Prepare adapter ligation mix based on the below table. Briefly centrifuge, mix by pipetting up and down 10-12 times and briefly centrifuge again.

| Component | Volume/reaction |
|---|---|
| Fragmentation, end-repair and A-addition reaction | 25 μl |
| Ligation buffer, 5x | 10 μl |
| IL-N7## adapter | 0.5 μl |
| DNA ligase | 5 μl |
| Ligation solution | 7.2 μl |
| Nuclease-free water | 2.3 μl |
| Total | 50 μl |

2. Program a thermal cycler to 20 degree Celsius and incubate the reactions for 15 minutes.

Cleanup of Adapter Ligated DNA
1. Add 30 μL nuclease-free water to bring each sample to 80 μL
2. Add 1124, QIAseq Beads. Mix well by pipetting up and down several times.
3. Incubate for 5 min at room temperature
4. Place the plate on a magnetic rack for 10 min. Carefully remove and discard the supernatant while beads still on magnetic stand.
5. Remove the plate from the magnetic rack and elute DNA from the beads by adding 52 μL nuclease-free water. Mix well by pipetting.
6. Return plate to magnetic rack until solution is cleared.
7. Transfer 50 μL of supernatant to clean plate.
8. Add 70 μL QIAseq Beads. Mix well by pipetting up and down.
9. Incubate for 5 min at room temperature
10. Place the plate on a magnetic rack for 5 minutes. With beads on the magnetic stand, carefully remove and discard the supernatant.
11. Add 200 μL 80% ethanol. Move the plate side-to-side between the two column positions of the magnet to wash the beads. Carefully remove and discard the wash.
12. Repeat ethanol wash.
13. With plate still on the magnetic stand, air dry at room temperature for 15 minutes.
14. Remove the plate from the magnetic stand, elute the DNA from the beads by adding 12 μL nuclease-free water. Mix well by pipetting.
15. Return plate to the magnetic rack until the solution has cleared.
16. Transfer 9.4 μL of the supernatant to clean tubes or plate.

Target Enrichment
1. Briefly centrifuge, prepare target enrichment mix by pipetting up and down 7-8 times and briefly centrifuge again.

| Component | Volume/reaction |
|---|---|
| Adapter-ligated DNA from | 9.4 μl |
| ‘Cleanup of adapter-ligated DNA’ | |
| TEPCR buffer, 5x | 4 μl |
| QIA_{seq} targeted DNA panel | 5 μl |
| IL-Forward primer | 0.8 μl |
| HotStarTaq DNA polymerase | 0.8 μl |
| Total | 20 μl |

2. Program a thermal cycler using the cycling conditions in Table 14 (panel with <1500 primers/tube) or Table 15 (panel with ≥1500 primers/tube).

TABLE 14
Cycling conditions for target enrichment
if number of primers <1500/tube

| Step | Time | Temperature |
|---|---|---|
| Initial denaturation | 13 min | 95° C. |
| | 2 min | 98° C. |
| 8 cycles | 15 s | 98° C. |
| | 10 min | 68° C. |
| 1 cycle | 5 min | 72° C. |
| Hold | 5 min | 4° C. |
| Hold | ∞ | 4° C. |

TABLE 15
Cycling conditions for target enrichment
if number of primers ≥1500/tube

| | Time | | |
|---|---|---|---|
| | (1500-12,000 | Time | |
| Step | primers/tube) | (>12,000 primers/tube) | Temperature |
| Initial | 13 min | 13 min | 95° C. |
| denaturation | 2 min | 2 min | 98° C. |
| 6 cycles | 15 s | 15 s | 98° C. |
| | 15 min | 30 min | 65° C. |
| 1 cycle | 5 min | 5 min | 72° C. |
| Hold | 5 min | 5 min | 4° C. |
| Hold | ∞ | ∞ | 4° C. |

3. Place the target enrichment reaction in the thermal cycler and start the run.
4. After the reaction is complete, place the reactions on ice and proceed with “Cleanup of target enrichment”, below. Alternatively, the samples can be stored at −20° C. in a constant-temperature freezer for up to 3 days.

Cleanup of Target Enriched DNA
1. Add 70 μl nuclease-free water to bring each sample to 90 μl.
2. Add 108 μl QIAseq Beads. Mix well by pipetting up and down several times.
3. Incubate for 5 min at room temperature.
4. Place the tubes/plate on a magnetic rack for 5 min. After the solution has cleared, with the beads still on the magnetic stand, carefully remove and discard the supernatant.
5. With the beads still on the magnetic stand, add 200 μl 80% ethanol. Rotate the tube (2-3 times) or move the plate side-to-side between the two column positions of the magnet to wash the beads. Carefully remove and discard the wash.
6. Repeat the ethanol wash.
7. With the beads still on the magnetic stand, air dry at room temperature for 10 min.
8. Remove the beads from the magnetic stand, and elute the DNA from the beads by adding 16 μl nuclease-free water. Mix well by pipetting.
9. Return the tube/plate to the magnetic rack until the solution has cleared.
10. Transfer 13.4 μl of the supernatant to clean tubes/plate.
11. Proceed with “Universal PCR”, below. Alternatively, the samples can be stored at −20° C. in a constant-temperature freezer for up to 3 days.

Universal PCR
1. Prepare the Universal PCR master mix (or reaction) according to Table 14 or Table 15, depending in which index set is being used. Briefly centrifuge, mix by pipetting up and down 7-8 times and briefly centrifuge again.
   Important: If using QIAseq 96-index I Set A, B, C or D, mix components directly in IL-S5 Index Primer Plate A, B, C or D that contains pre-dispensed, dried index primer and Universal PCR primers.
   Important: The A, B, C or D IL-N7 Adapter Plate used in the adapter ligation reaction must be paired with the matching A, B, C or D IL-S5 Index Primer Plate used in the Universal PCR amplification reaction.

Reaction mix for Universal PCR if using QIAseq 12-index I

| Component | Volume/reaction |
|---|---|
| Target-enriched DNA from | 13.4 μl |
| ‘Cleanup of target enrichment’ | |
| UPCR buffer, 5x | 4 μl |
| IL-Universal primer | 0.8 μl |
| IL-S502 index primer | 0.8 μl |
| HotStarTaq DNA polymerase | 1 μl |
| Total | 20 μl |

Reaction Components for Universal PCR if using QIAseq 96-index I Set A, B, C or D*

| Component | Volume/reaction |
|---|---|
| Target-enriched DNA from | 13.4 μl |
| ‘Cleanup of target enrichment’ | |
| UPCR buffer, 5x | 4 μl |
| HotStarTaq DNA polymerase | 1 μl |
| Nuclease-free water | 1.6 μl |
| Total | 20 μl |

Applies to QIAseq IL-55 Index Primer Plate in A, B, C or D set. The final library dual sample index is determined by the combination of the IL-N7 Adapter Plate and the QIAseq IL-55 Index Primer Plate. Total sample index level can be up to 384-plex if using QIAseq 96-index A, B, C and D sets together.
2. Program a thermal cycler using the cycling conditions in Table 16 (cycling program) and Table 17 (cycle number).

TABLE 16
Cycling conditions for Universal PCR

| Step | Time | Temperature |
|---|---|---|
| Initial denaturation | 13 min | 95° C. |
| | 2 min | 98° C. |
| Number of cycles | 15 sec | 98° C. |
| (see Table 17) | 2 min | 60° C. |
| 1 cycle | 5 min | 72° C. |
| Hold | 5 min | 4° C. |
| Hold | ∞ | 4° C. |

TABLE 17
Amplification cycles for Universal PCR

| Primers per pool | Cycle number |
|---|---|
| 6-24 | 28 |
| 25-96 | 26 |
| 97-288 | 24 |
| 289-1056 | 23 |
| 1057-1499 | 22 |
| 1500-3072 | 23 |
| 3073-4999 | 22 |
| 5000-12,000 | 21 |
| ≥12,001 | 20 |

3. After the reaction is complete, place the reactions on ice and proceed to “Cleanup of Universal PCR”. Alternatively, the samples can be stored at ˜20° C. in a constant-temperature freezer for up to 3 days.

Cleanup of Universal PCR
1. For cfDNA samples, add 70 μl nuclease-free water to bring each sample to 90 μl.
2. For cfDNA samples, add 108 μl QIAseq Beads. Mix well by pipetting up and down several times.
3. Incubate for 5 min at room temperature.
4. Place the tubes/plate on magnetic rack for 5 min to separate beads from supernatant. Once the solution has cleared, with the beads still on the magnetic stand, carefully remove and discard the supernatant. (Important: Do not discard the beads as they contain the DNA of interest.)
5. With the beads still on the magnetic stand, add 200 μl 80% ethanol. Rotate the tube (2-3 times) or move the plate side-to-side between the two column positions of the magnet to wash the beads. Carefully remove and discard the wash.
6. Repeat the ethanol wash. (Important: Completely remove all traces of the ethanol wash after this second wash. Remove the ethanol with a 200 μl pipet first, and then use a 10 μl pipet to remove any residual ethanol.)
7. With the beads still on the magnetic stand, air dry at room temperature for 10 min. (Note: Visually inspect that the pellet is completely dry. Remove the beads from the magnetic stand, and elute the DNA from the beads by adding 30 μl nuclease-free water. Mix well by pipetting.)
8. Return the tubes/plate to the magnetic rack until the solution has cleared.
9. Transfer 28 μl supernatant to clean tubes or plate.
10. The library can be stored in a ˜20° C. freezer prior to quantification using the QIAseq.

### Example 4

### Aneuploidy Determination

This example illustrates one embodiment of determination of aneuploidy using one embodiment of the present invention.

Tables 18, 19 and 20 present the data of the present aneuploidy test in three independent runs that delivered positive results.

As shown in Table 18, sample no. “12_S12A” returned a “maternal T21 positive” result, meaning that the fetus has a high likelihood in having a maternal trisomy 21.

The performance of the present aneuploidy test may be affected if the quality control at various steps of plasma sample handling, DNA extraction and library preparation are inadequate. The collection, storage and processing of whole blood; extraction of cfDNA; selection of extracted cfDNA for library construction; library DNA quality for adapter ligation; appropriate library fragment size; appropriate ratio for target-capture hybridization; hybridization condition for optimal target enrichment and final pooling of libraries for loading onto the sequencer are some of the factors that may affect the performance of the sequencing and the test results.

TABLE 18
Data from one run with one positive result (No. 1)

| | | | SNP | Fetal | |
|---|---|---|---|---|---|
| Sample | Read pairs | Coverage | coverage | fraction | Result |
| 01_S1A | 38097224 | 182.86 | 199.77 | 4.7% | negative |
| 02_S2A | 45196372 | 256.48 | 281.59 | 8.2% | negative |
| 03_S3A | 43634203 | 168.15 | 183.05 | 6.9% | negative |
| 04_S4A | 42638863 | 260.33 | 286.59 | 15.5% | negative |
| 05_S5A | 44825373 | 244.05 | 267.86 | 6.8% | negative |
| 06_S6A | 43867110 | 207.22 | 227.4 | 24.5% | negative |
| 07_S7A | 55238767 | 228.12 | 248.54 | 9.9% | negative |
| 08_S8A | 27812487 | 128.92 | 140.83 | 3.6% | negative |
| 09_S9A | 40747397 | 161.32 | 175.66 | 3.3% | negative |
| 10_S10A | 32186120 | 161.98 | 177.48 | 11.9% | negative |
| 11_S11A | 43451884 | 201.57 | 220.13 | 9.8% | negative |
| 12_S12A | 38878996 | 228.05 | 249.94 | 12.8% | maternal T21 |
| | | | | | positive |

TABLE 19
Data from one run with two positive results (No. 2)

| | | | SNP | Fetal | |
|---|---|---|---|---|---|
| Sample | Read pairs | Coverage | coverage | fraction | Result |
| 01_S1B | 42306959 | 296.64 | 324.3 | 12.7% | maternal T21 |
| | | | | | positive |
| 02_S2B | 38755027 | 211.25 | 230.28 | 9.9% | negative |
| 03_S3B | 38396370 | 195 | 212.67 | 11.8% | negative |
| 04_S4B | 38736195 | 169.43 | 183.72 | 3.5% | negative |
| 05_S5B | 35829073 | 160.24 | 173.74 | 3.8% | negative |
| 06_S6B | 39280785 | 214.21 | 233.51 | 10.2% | negative |
| 07_S7B | 36602096 | 210.08 | 229.32 | 14.4% | maternal T21 |
| | | | | | positive |
| 08_S8B | 41807037 | 270.69 | 296.95 | 6.8% | negative |
| 09_S9B | 40565843 | 299.25 | 328.96 | 15.3% | negative |
| 10_S10B | 32272523 | 183.85 | 200.74 | 7.0% | negative |
| 11_S11B | 39840308 | 301.33 | 330.69 | 8.2% | negative |
| 12_S12B | 35343370 | 209.18 | 228.65 | 4.7% | negative |

TABLE 20
Data from one run with one positive result (No. 3)

| | | | SNP | Fetal | |
|---|---|---|---|---|---|
| Sample | Read pairs | Coverage | coverage | fraction | Result |
| 01_S1C | 28814025 | 206.46 | 221.46 | 8.3% | negative |
| 02_S2C | 28590881 | 206.32 | 221.81 | 13.0% | negative |
| 03_S3C | 17032658 | 124.64 | 134.12 | 7.2% | negative |
| 04_S4C | 31993771 | 233.42 | 250.48 | 16.5% | negative |
| 05_S5C | 37321674 | 290.94 | 313.36 | 13.2% | negative |
| 06_S6C | 24551472 | 185.86 | 200.16 | 13.9% | negative |
| 07_S7C | 36133862 | 268.6 | 287.71 | 2.2% | negative |
| 08_S8C | 28424359 | 210.01 | 225.32 | 3.3% | negative |
| 09_S9C | 22147900 | 162.86 | 175.02 | 11.5% | negative |
| 10_S10C | 17868313 | 139.48 | 149.93 | 8.5% | negative |
| 11_S11C | 28317548 | 207.02 | 222.01 | 5.8% | negative |
| 12_S12C | 17491807 | 126.46 | 136.04 | 11.9% | maternal T13 |
| | | | | | positive |

### REFERENCES

1. Goya. Rodrigo, et al. “SNVMix: predicting single nucleotide variants from next-generation sequencing of tumors.” Bioinformatics 26.6 (2010): 730-736.
2. John Besser, Heather A. Carleton, Peter Gerner-Smidt, Rebecca L. Lindsey, and Eija Trees. “Next-Generation Sequencing Technologies and their Application to the Study and Control of Bacterial Infections”, Clin Microbiol Infect. 2018 April; 24(4): 335-341.
3. Jiang, Peiyong, et al. “FetalQuant: deducing fractional fetal DNA concentration from massively parallel sequencing of DNA in maternal plasma.” Bioinformatics 28.22 (2012): 2883-2890.
4. Hassold, Terry, and Patricia Hunt. “To err (meiotically) is human: the genesis of human aneuploidy.” Nature Reviews Genetics 2.4 (2001): 280.
5. [symbol] [symbol] [symbol] , 2008-2013 [symbol] [symbol] [symbol]
6. Sahlin, Ellika, et al. “Molecular and cytogenetic analysis in stillbirth: results from 481 consecutive cases,” Fetal diagnosis and therapy 36.4 (2014): 326-332,
7. Zhang, Xiao-Hui, et al. “Chromosomal abnormalities: subgroup analysis by maternal age and perinatal features in Zhejiang province of China, 2011-2015,” Italian journal of pediatrics 43.1 (2017): 47.
8. [symbol] ⋅ [symbol] ⋅ [symbol] ⋅ [symbol] ⋅ [symbol] ⋅ . . . , 1713 [symbol] [symbol] [symbol]
9. Curnow, Kirsten J., et al. “Clinical experience and follow-up with large scale single-nucleotide polymorphism-based noninvasive prenatal aneuploidy testing,” American Journal of Obstetrics Gynecology 211.5 (2014): 527-e1.

1. A method for determining the probability that a fetus suffers from aneuploidy, comprising:
a) obtaining a test sample from a pregnant woman carrying the fetus, the sample comprising cell-free fetal DNA and cell-free maternal DNA;
b) enriching a plurality of target sequences in the cell-free fetal DNA and cell-free maternal DNA, the target sequences comprising a plurality of biallelic autosomal single nucleotide polymorphisms (SNPs) of interest;
c) amplifying the enriched target sequences, thereby obtaining amplified target sequences;
d) determining the sequence of at least a portion of some or all of the amplified target sequences, wherein the portion encompasses at least one biallelic autosomal SNP of interest; and
e) determining the probability that the fetus suffers from aneuploidy by analyzing allele frequencies of the at least one biallelic autosomal SNP of interest using an expectation-maximization algorithm module, a total probability module and a Bayesian module.

2. The method of claim 1, wherein b) further comprises amplifying at least some of the target sequences.

3. The method of claim 1, wherein b) further comprises capturing at least some of the target sequences by probe hybridization.

4. The method of claim 1, wherein the test sample is derived from a blood sample from a pregnant woman.

5. The method of claim 1, wherein the SNPs of interest are SNPs located on the same chromosome, wherein an abnormal copy number of the chromosome causes the aneuploidy.

6. The method of claim 1, wherein the method determines the probability of two or more types of aneuploidy from which the fetus suffers, wherein a different chromosome is responsible for each of the two or more types of aneuploidy.

7. The method of claim 1, wherein the aneuploidy is selected from the group consisting of trisomy 13, trisomy 18 and trisomy 21.

8. The method of claim 1, wherein d) is performed using a platform capable of next-generation sequencing.

9. The method of claim 1, wherein e) comprises:
i. determining the largest likelihoods of euploidy and of aneuploidy using the expectation-maximization algorithm module;
ii. determining prior probabilities of euploidy and of aneuploidy from a plurality of conditional probabilities using the total probability module; and
iii. transforming the determined largest likelihoods of euploidy and of aneuploidy and the determined prior probabilities of euploidy and of aneuploidy to posterior probabilities of euploidy and of aneuploidy in the fetus using the Bayesian module.

10. The method of claim 9, wherein the conditional probabilities comprise conditional probabilities of aneuploidy based on survival probabilities of fetuses and conditional probabilities of survival of fetuses based on maternal age and gestational week, wherein the conditional probabilities are not specific to the fetus in question.

11. The method of claim 9, wherein the largest likelihoods of a) are determined based on the allele count, mapping quality and base quality of the reference allele and alternative allele for a given SNP.

12. The method of claim 1, further comprising determining whether the fetus has an aneuploidy by comparing the determined probability of aneuploidy and a cutoff value which produces a pre-determined sensitivity.

13. A method for determining the probability that a fetus suffers from aneuploidy, comprising:
a) obtaining a blood sample from a pregnant woman carrying the fetus;
b) extracting from the sample cell-free fetal DNA and cell-free maternal DNA to form a test sample;
c) determining the concentration of cell-free DNA in the test sample;
d) preparing from the test sample a library of nucleic acids comprising a plurality of target sequences, the target sequences comprising a plurality of biallelic autosomal single nucleotide polymorphisms (SNPs) of interest;
e) sequencing at least a portion of the library; and
f) determining the probability that the fetus suffers from aneuploidy by analyzing allele frequencies in the plurality of SNPs using an expectation-maximization algorithm module, a total probability module and a Bayesian module.

14. The method of claim 13, wherein d) comprises enriching the target sequences from the cell-free DNA.

15. The method of claim 14, wherein enriching the target sequences from the cell-free DNA comprises amplifying at least a portion of the target sequences.

16. The method of claim 14, wherein enriching the target sequences from the cell-free DNA comprises capturing at least a portion of the target sequences by probe hybridization.

17. The method of claim 13, wherein d) comprises:
i. end-repairing and A-tailing of the cell-free DNA;
ii. ligating the cell-free fetal DNA obtained from (i) with adapters, thereby obtaining ligated DNA sequences;
iii. amplifying the ligated DNA sequences;
iv. hybridizing the DNA sequences from (iii) with probes comprising sequences that are specific to the target sequences, thereby capturing DNA sequences comprising the target sequences; and
v. amplifying the captured DNA sequences, thereby obtaining a plurality of DNA sequences comprising the target sequences.

18. The method of claim 13, wherein d) comprises:
i. end-repairing and A-tailing of the cell-free DNA;
ii. ligating the cell-free DNA obtained from (i) with adapters, thereby obtaining ligated DNA sequences; and
iii. amplifying the ligated DNA sequences using primers specific to the target sequences, thereby obtaining a plurality of amplicons; and
iv. amplifying the plurality of amplicons, thereby obtaining a plurality of DNA sequences comprising the target sequences.

19. The method of claim 13, wherein:
i. the SNPs of interest are SNPs located on the same chromosome, wherein an abnormal copy number of the chromosome causes the aneuploidy;
ii. the method determines the probability of two or more types of aneuploidy from which the fetus suffers, wherein a different chromosome is responsible for each of the two or more types of aneuploidy; or
iii. the aneuploidy is selected from the group consisting of trisomy 13, trisomy 18 and trisomy 21.

20.-21. (canceled)

22. The method of claim 13, wherein e) is performed using a platform capable of next-generation sequencing.

23. The method of claim 13, wherein f) further comprises:
i. determining largest likelihoods of euploidy and of aneuploidy using the expectation-maximization algorithm module;
ii. determining prior probabilities of euploidy and aneuploidy from a plurality of conditional probabilities using the total probability module; and
iii. transforming the determined largest likelihoods of euploidy and aneuploidy and the determined prior probabilities of euploidy and of aneuploidy to posterior probabilities of aneuploidy in the fetus using the Bayesian module.

24. The method of claim 23, wherein the conditional probabilities comprise conditional probabilities of aneuploidy based on survival probabilities of fetuses and conditional probabilities of survival of fetuses based on maternal age and gestational week, wherein the conditional probabilities are not specific to the fetus in question.

25. The method of claim 23, wherein the largest likelihoods of a) are determined based on the allele count, mapping quality and base quality of the reference allele and alternative allele for a given SNP.

26. The method of claim 13, further comprising determining whether the fetus has an aneuploidy by comparing the determined probability of aneuploidy and a cutoff value which produces a pre-determined sensitivity.

27. A system for determining the probability of an aneuploidy in a fetus based on genetic data from a blood sample of a pregnant woman carrying the fetus, wherein the blood sample comprises a mixture of nucleic acids from the woman and the fetus, comprising
i. a means for receiving the genetic data from the sample, wherein the genetic data comprises information about a plurality of biallelic autosomal single nucleotide polymorphisms (SNPs) of interest;
ii. an expectation-maximization algorithm module for determining largest likelihoods of euploidy and of aneuploidy, thereby generating a likelihood ratio;
iii. a total probability module for determining prior probabilities of euploidy and of aneuploidy from a plurality of conditional probabilities; and
iv. a Bayesian module for transforming the determined likelihood ratio and the determined prior probabilities of euploidy and of aneuploidy to posterior probabilities of euploidy and of aneuploidy, wherein the posterior probabilities yield the probability of aneuploidy in the fetus.

28. The system of claim 27, wherein the genetic data:
i. comprises allele count, mapping quality and base quality of the reference allele and alternative allele for a given SNP; and
ii. are derived from a library of DNA sequences comprising the SNPs of interest.

29. (canceled)

30. The system of claim 27, wherein the genetic data are derived from data obtained from a platform capable of next-generation sequencing.

31. The system of claim 27, wherein the conditional probabilities comprise conditional probabilities of aneuploidy based on survival probabilities of fetuses and conditional probabilities of survival of fetuses based on maternal age and gestational week, wherein the conditional probabilities are not specific to the fetus in question.

32. The system of claim 27, wherein:
i. the SNPs of interest are SNPs located on the same chromosome, wherein an abnormal copy number of the chromosome causes the aneuploidy;
ii. the method determines the probability of two or more types of aneuploidy from which the fetus suffers, wherein a different chromosome is responsible for each of the two or more types of aneuploidy; or
iii. the aneuploidy is selected from the group consisting of trisomy 13, trisomy 18 and trisomy 21.

33.-34. (canceled)

35. Use of the system of claim 27 for determining the probability of an aneuploidy in a fetus.
